(19) United States
(12) Patent Application Publication
(10) Pub. No.: US 2025/0032023 A1
(43) Pub. Date: Jan. 30, 2025

(54) MUSCLE STRENGTH EVALUATION DEVICE, MUSCLE STRENGTH EVALUATION SYSTEM, MUSCLE STRENGTH EVALUATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,325

(22) PCT Filed: Dec. 27, 2021
(86) PCT No.: PCT/JP2021/048564

§ 371 Date: Jun. 4, 2024

(51) Int. Cl. A61B 5/22; A61B 5/11; G16H 50/30

(52) U.S. Cl. CPC A61B 5/224; A61B 5/112; G16H 50/30

(57) ABSTRACT

Provided is a muscle strength evaluation device that includes a data acquisition unit that acquires feature quantity data including a feature quantity used for estimation of a muscle strength of an evaluation target muscle related to a fall risk, the feature quantity data being extracted from sensor data related to movement of a user's foot, a storage unit that stores an estimation model that outputs a muscle strength index of the evaluation target muscle according to an input of the feature quantity data, an evaluation unit that inputs the acquired feature quantity data to the estimation model and evaluate a muscle strength of the evaluation target muscle of the user according to the muscle strength index output from the estimation model, and an output unit that outputs information regarding an evaluation result related to the muscle strength of the evaluation target muscle of the user.

### TECHNICAL FIELD

The present disclosure relates to a muscle strength evaluation device and the like for evaluating muscle strength using sensor data related to movement of a foot.

### BACKGROUND ART

With increasing interest in healthcare, services that provide information corresponding to features (also referred to as gait) included in a walking pattern have attracted attention. For example, a technique for analyzing a gait based on sensor data measured by a sensor mounted on footwear such as shoes has been developed. In the time-series data of the sensor data, a feature of a gait event (also referred to as a walking event) related to a physical condition appears.

PTL 1 discloses a device that detects an abnormality of a foot based on features of walking of a pedestrian. The device of PTL 1 extracts a characteristic walking feature quantity in walking of a pedestrian wearing footwear by using data acquired from a sensor installed on the footwear. The device of PTL 1 detects an abnormality of a pedestrian walking while wearing footwear based on the extracted walking feature quantity. For example, the device of PTL 1 extracts a characteristic site regarding hallux valgus from walking waveform data for one gait cycle. The device of PTL 1 estimates the progress state of hallux valgus using the walking feature quantity of the extracted characteristic site.

Fall can cause various injuries to elderly people. Decrease in physical ability, such as lowering in muscle strength, can be a factor in the risk of falling. Factors of lowering in muscle strength vary among individuals. Therefore, it is necessary to take measures against lowering in muscle strength tailored to the individual. If muscles related to walking can be evaluated according to the gait, training leading to reduction in the risk of falling can be recommended according to the individual.

PTL 2 discloses a training support system in which an expert gives a personal guidance to a user performing training at home. The system of PTL 2 stores the test result of the physical ability of the user, the user identification information, and the time information related to the test implementation in association with each other. The system of PTL 2 evaluates variations among a plurality of test results with respect to time information related to a plurality of test implementations. The system of PTL 2 notifies that training information should be updated based on a variation among the test results.

### CITATION LIST

### Patent Literature

PTL 1: WO 2021/140658 A1
PTL 2: JP 2013-066672 A

### SUMMARY OF INVENTION

### Technical Problem

In the method of PTL 1, the progress state of hallux valgus is estimated using the walking feature quantity of the characteristic site extracted from the data acquired from the sensor installed on the footwear. PTL 1 does not disclose estimating the easy-falling property by using the walking feature quantity of the characteristic site extracted from the data acquired from the sensor installed on the footwear.

According to the method of PTL 2, training according to decrease in physical ability of the user can be prescribed. However, the method of PTL 2 cannot specify a muscle that is a factor of decrease in physical ability. Therefore, in the method of PTL 2, appropriate training for training the muscle, which is a factor of decrease in physical ability, cannot be prescribed.

An object of the present disclosure is to provide a muscle strength evaluation device and the like capable of evaluating muscle strength of a muscle related to a risk of falling according to a gait in daily life.

### Solution to Problem

A muscle strength evaluation device according to one aspect of the present disclosure includes a data acquisition unit that acquires feature quantity data including a feature quantity used for estimation of a muscle strength of an evaluation target muscle related to a fall risk, the feature quantity data being extracted from sensor data related to movement of a user's foot, a storage unit that stores an estimation model that outputs a muscle strength index of the evaluation target muscle according to an input of the feature quantity data, an evaluation unit that inputs the acquired feature quantity data to the estimation model and evaluate a muscle strength of the evaluation target muscle of the user according to the muscle strength index output from the estimation model, and an output unit that outputs information regarding an evaluation result related to the muscle strength of the evaluation target muscle of the user.

A muscle strength evaluation method according to one aspect of the present disclosure includes acquiring feature quantity data including a feature quantity used for estimation of muscle strength of an evaluation target muscle related to a fall risk, the feature quantity data being extracted from sensor data regarding a movement of a user's foot, inputting the acquired feature quantity data to an estimation model that outputs a muscle strength index of the evaluation target muscle according to an input of the feature quantity data, evaluating the muscle strength of the evaluation target muscle of the user according to the muscle strength index output from the estimation model, and outputting information regarding an evaluation result related to a muscle strength of the evaluation target muscle of the user.

A program according for causing a computer to execute processes of acquiring feature quantity data including a feature quantity used for estimation of muscle strength of an evaluation target muscle related to a fall risk, the feature quantity data being extracted from sensor data regarding a movement of a user's foot, inputting the acquired feature quantity data to an estimation model that outputs a muscle strength index of the evaluation target muscle according to an input of the feature quantity data, evaluating the muscle strength of the evaluation target muscle of the user according to the muscle strength index output from the estimation model, and outputting information regarding an evaluation result related to a muscle strength of the evaluation target muscle of the user.

### Advantageous Effects of Invention

According to the present disclosure, an object of the present disclosure can provide a muscle strength evaluation device and the like capable of evaluating muscle strength of a muscle related to a risk of 5 falling according to a gait in daily life.

### BRIEF DESCRIPTION OF DRAWINGS

FIG. 1 is a block diagram illustrating an example of a configuration of a muscle strength evaluation system according to a first example embodiment.

FIG. 2 is a block diagram illustrating an example of a configuration of a gait measuring device included in the muscle strength evaluation system according to the first example embodiment.

FIG. 3 is a conceptual diagram illustrating an arrangement example of the gait measuring device according to the first example embodiment.

FIG. 4 is a conceptual diagram for describing an example of a relationship between a local coordinate system and a world coordinate system set in the gait measuring device according to the first example embodiment. 20

FIG. 5 is a conceptual diagram for describing a human body surface used in the description regarding the gait measuring device according to the first example embodiment.

FIG. 6 is a conceptual diagram for describing a gait cycle used in the description regarding the gait measuring device according to the first example embodiment.

FIG. 7 is a conceptual diagram for describing a gait parameter used in the description regarding the gait measuring device according to the first example embodiment.

FIG. 8 is a graph for describing an example of time-series data of sensor data measured by the gait measuring device according to the first example embodiment.

FIG. 9 is a diagram for describing an example of normalization of walking waveform data extracted from time-series data of the sensor data measured by the gait measuring device according to the first example embodiment.

FIG. 10 is a conceptual diagram for describing an example of a walking phase cluster from which the feature quantity data generating device of the gait measuring device according to the first example embodiment extracts a feature quantity.

FIG. 11 is a block diagram showing an example of a configuration of a muscle strength evaluation device included in the muscle strength evaluation system according to the first example embodiment.

FIG. 12 is a conceptual diagram for explaining items related to an evaluation target muscle of the muscle strength evaluation system according to the first example embodiment.

FIG. 13 is a table summarizing feature quantities regarding total muscle strength (grip strength) of the whole body related to an evaluation target muscle of the muscle strength evaluation system according to the first example embodiment.

FIG. 14 is a table summarizing feature quantities related to dynamic balance related to an evaluation target muscle of the muscle strength evaluation system according to the first example embodiment.

FIG. 15 is a table summarizing feature quantities regarding lower limb muscle strength related to an evaluation target muscle of the muscle strength evaluation system according to the first example embodiment.

FIG. 16 is a table summarizing feature quantities regarding mobility related to an evaluation target muscle of the muscle strength evaluation system according to the first example embodiment.

FIG. 17 is a table summarizing feature quantities related to static balance related to an evaluation target muscle of the muscle strength evaluation system according to the first example embodiment.

FIG. 18 is a conceptual diagram for explaining an example of an evaluation target muscle by the muscle strength evaluation device included in the muscle strength evaluation system according to the first example embodiment.

FIG. 19 is a conceptual diagram illustrating an estimation example of a muscle strength score (muscle strength index) by the muscle strength evaluation device included in the muscle strength evaluation system according to the first example embodiment.

FIG. 20 is a flowchart for explaining an example of the operation of the gait measuring device included in the muscle strength evaluation system according to the first example embodiment.

FIG. 21 is a flowchart for explaining an example of the operation of the muscle strength evaluation device included in the muscle strength evaluation system according to the first example embodiment.

FIG. 22 is a conceptual diagram for explaining an application example of the muscle strength evaluation system according to the first example embodiment.

FIG. 23 is a conceptual diagram for explaining an application example of the muscle strength evaluation system according to the first example embodiment.

FIG. 24 is a conceptual diagram for explaining an application example of the muscle strength evaluation system according to the first example embodiment.

FIG. 25 is a block diagram illustrating an example of a configuration of a machine learning system according to a second example embodiment.

FIG. 26 is a block diagram illustrating an example of a configuration of a machine learning device included in the machine learning system according to the second example embodiment.

FIG. 27 is a conceptual diagram for explaining an example of machine learning by a machine learning device included in the machine learning system according to the second example embodiment.

FIG. 28 is a conceptual diagram for explaining an example of machine learning by a machine learning device included in the machine learning system according to the second example embodiment.

FIG. 29 is a block diagram showing an example of a configuration of a muscle strength evaluation device according to a third example embodiment.

FIG. 30 is a block illustrating an example of a hardware configuration that executes control and process of each example embodiment.

### EXAMPLE EMBODIMENT

Hereinafter, example embodiments of the present invention will be described with reference to the drawings. However, the example embodiments described below have technically preferable limitations for carrying out the present invention, but the scope of the invention is not limited to the following. In all the drawings used in the following description of the example embodiments, the same reference numerals are given to the same parts unless there is a particular reason. Furthermore, in the following example embodiments, repeated description of similar configurations and operations may be omitted.

### First Example Embodiment

First, a muscle strength evaluation system according to a first example embodiment will be described with reference to the drawings.

The muscle strength evaluation system of the present example embodiment measures sensor data related to the movement of the foot corresponding to the walking of the user. The muscle strength evaluation system of the present example embodiment estimates the muscle strength of the user using the measured sensor data.

In the present example embodiment, an example will be given in which the muscle strength is estimated according to the relevance between the feature (also referred to as gait) included in the walking pattern and the muscle (also referred to as evaluation target muscle) related to the risk The muscle strength of the evaluation target muscle can be of falling. evaluated based on the variability of the gait parameters. In the present example embodiment, muscle strength is estimated based on five related items (also referred to as five items) related to gait. The five items relate to total muscle strength (grip strength) of the whole body, dynamic balance, lower limb muscle strength, mobility, and static balance. These five items have a correlation with muscle strength. The five items are related to each other to some extent, but can be regarded as basically independent. In the present example embodiment, an example of estimating muscle strength based on all five items will be mainly described. The muscle strength can be estimated based on at least one of the five items. The lowering in muscle strength associated with the five items becomes a factor that increases the risk of falling.

### (Configuration)

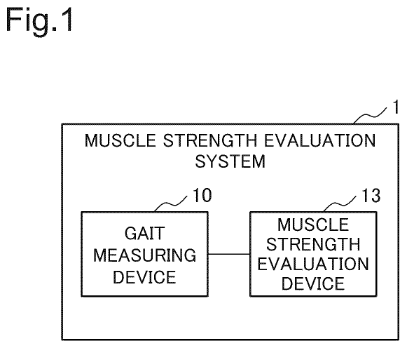

FIG. 1 is a block diagram illustrating an example of a configuration of a muscle strength evaluation system 1 according to the present example embodiment. The muscle strength evaluation system 1 includes a gait measuring device 10 and a muscle strength evaluation device 13. In the present example embodiment, an example in which the gait measuring device 10 and the muscle strength evaluation device 13 are configured as separate hardware will be described. For example, the gait measuring device 10 is installed on footwear or the like of a subject (user) whose muscle strength is to be estimated. For example, the function of the muscle strength evaluation device 13 is installed in a mobile terminal carried by the subject (user). Hereinafter, configurations of the gait measuring device 10 and the muscle strength evaluation device 13 will be individually described.

### [Gait Measuring Device]

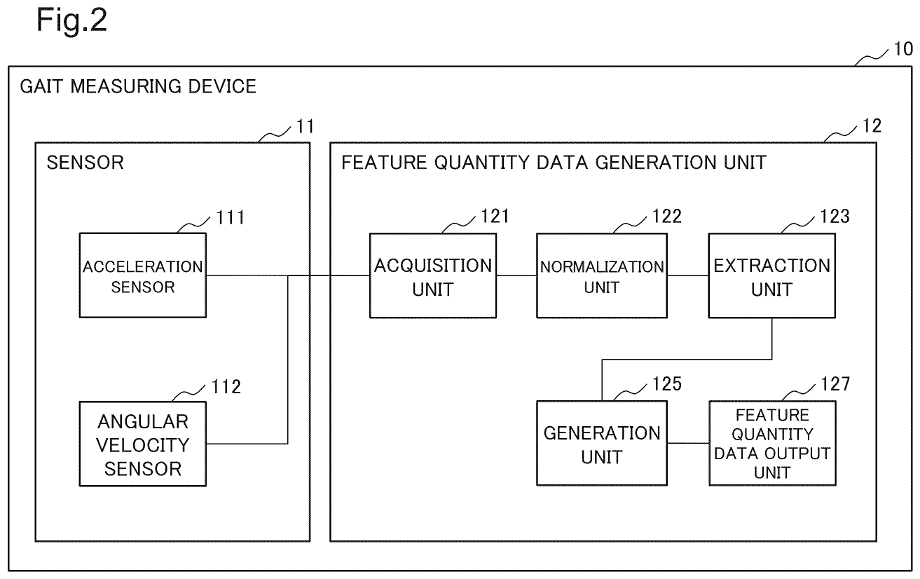

FIG. 2 is a block diagram illustrating an example of a configuration of the gait measuring device 10. The gait measuring device 10 includes a sensor 11 and a feature quantity data generation unit 12. In the present example embodiment, an example in which the sensor 11 and the feature quantity data generation unit 12 are integrated will be described. The sensor 11 and the feature quantity data generation unit 12 may be provided as separate devices.

As illustrated in FIG. 2, the sensor 11 includes an acceleration sensor 111 and an angular velocity sensor 112. FIG. 2 includes an example in which the acceleration sensor 111 and the angular velocity sensor 112 are included in the sensor 11. The sensor 11 may include a sensor other than the acceleration sensor 111 and the angular velocity sensor 112. Description on sensors other than the acceleration sensor 111 and the angular velocity sensor 112 that can be included in the sensor 11 will be omitted.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the feature quantity data generation unit 12. For example, a sensor of a piezoelectric type, a piezoresistive type, a capacitance type, or the like can be used as the acceleration sensor 111. The sensor used as the acceleration sensor 111 is not limited to the measurement method as long as the sensor can measure acceleration.

The angular velocity sensor 112 is a sensor that measures an angular velocity (also referred to as a spatial angular velocity) around three axes. The angular velocity sensor 112 measures an angular velocity (also referred to as a spatial angular velocity) as a physical quantity related to the movement of the foot. The angular velocity sensor 112 outputs the measured angular velocity to the feature quantity data generation unit 12. For example, a sensor of a vibration type, a capacitance type, or the like can be used as the angular velocity sensor 112. The sensor used as the angular velocity sensor 112 is not limited to the measurement method as long as the sensor can measure the angular velocity.

The sensor 11 is achieved by, for example, an inertial measuring device that measures acceleration and angular velocity. An example of the inertial measuring device may be an inertial measurement unit (IMU). The IMU includes an acceleration sensor 111 that measures acceleration in the three axes directions and an angular velocity sensor 112 that measures angular velocities around the three axes. The sensor 11 may be achieved by an inertial measuring device such as a vertical gyro (VG) or an attitude heading (AHRS). Furthermore, the sensor 11 may be achieved by Global Positioning System/Inertial Navigation System (GPS/INS). The sensor 11 may be achieved by a device other than the inertial measuring device as long as the sensor can measure a physical quantity related to the movement of the foot.

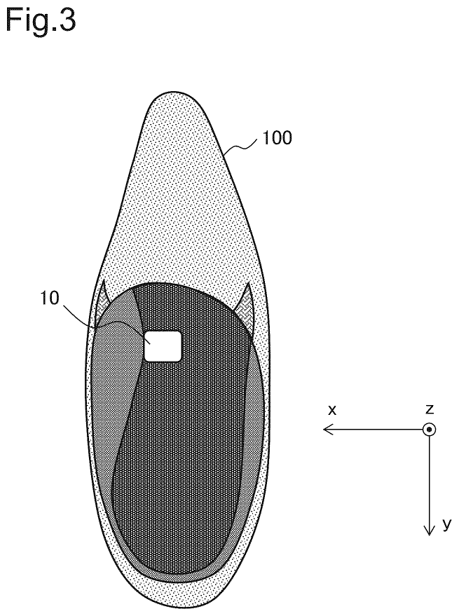

FIG. 3 is a conceptual diagram illustrating an example in which the gait measuring device 10 is arranged in the shoe 100 of the right foot. In the example of FIG. 3, the gait measuring device 10 is installed at a position corresponding to the back side of the foot arch. For example, the gait measuring device 10 is arranged in an insole inserted into the shoe 100. For example, the gait measuring device 10 may be arranged on the bottom surface of the shoe 100. For example, the gait measuring device 10 may be embedded in the main body of the shoe 100. The gait measuring device 10 may be detachable from the shoe 100 or may not be detachable from the shoe 100. The gait measuring device 10 may be installed at a position other than the back side of the foot arch as long as sensor data related to the movement of the foot can be measured. Furthermore, the gait measuring device 10 may be installed on a sock worn by the user or a decorative article such as an anklet worn by the user. Furthermore, the gait measuring device 10 may be directly attached to the foot or may be embedded in the foot. FIG. 3 illustrates an example in which the gait measuring device 10 is installed in the shoe 100 of the right foot. The gait measuring device 10 may be installed on the shoes 100 of both feet.

In the example of FIG. 3, a local coordinate system including an x axis in the left-right direction, a y axis in the front-back direction, and a z axis in the up-down direction is set with reference to the gait measuring device 10 (sensor 11). In the x axis, the left side is positive, in the y axis, the back side is positive, and in the z axis, the upper side is positive. The direction of the axis set in the sensor 11 may be the same for the left and right feet, or may be different for the left and right feet. For example, in a case where the sensors 11 produced with the same specifications are arranged in the left and right shoes 100, the up-down directions (direction in the Z axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same on the left and right.

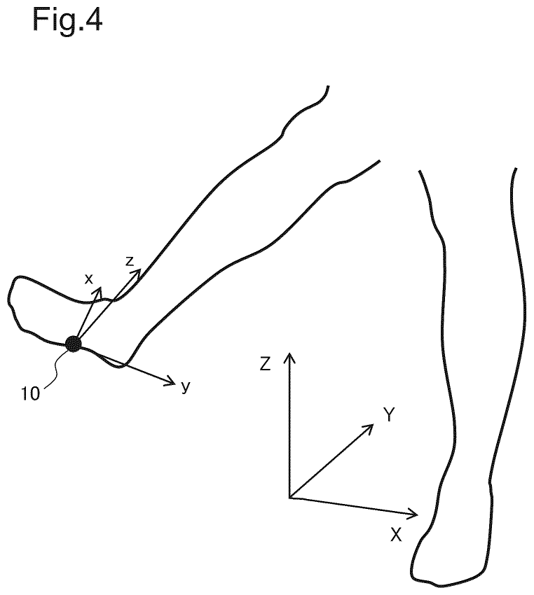

FIG. 4 is a conceptual diagram for describing a local coordinate system (x axis, y axis, z axis) set in the gait measuring device 10 (sensor 11) installed on the back side of the foot arch and a world coordinate system (X axis, Y axis, Z axis) set with respect to the ground. In the world coordinate system (X axis, Y axis, Z axis), in a state where the user facing the advancing direction is standing upright, the lateral direction of the user is set to the X axis direction (the leftward direction is positive), the direction of the back surface of the user is set to the Y axis direction (the backward direction is positive), and the gravity direction is set to the Z axis direction (the vertically upward direction is positive). The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies according to the walking of the user.

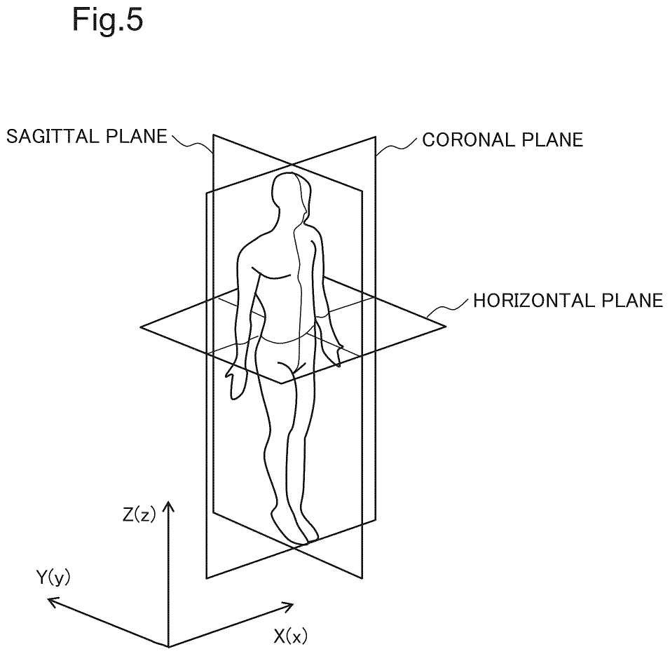

FIG. 5 is a conceptual diagram for describing a surface (also referred to as a human body surface) set with respect to the human body. In the present example embodiment, a sagittal plane dividing the body into left and right, a coronal plane dividing the body into front and back, and a horizontal plane dividing the body horizontally are defined. As illustrated in FIG. 5, the world coordinate system and the local coordinate system coincide with each other in a standing upright state in which the center line of the foot is oriented in the advancing direction. In the present example embodiment, rotation within the sagittal plane with the x axis as the rotation axis is defined as roll, rotation within the coronal plane with the y axis as the rotation axis is defined as pitch, and rotation within the horizontal plane with the z axis as the rotation axis is defined as yaw. In addition, a rotation angle within the sagittal plane with the x axis as a rotation axis is defined as a roll angle, a rotation angle within the coronal plane with the y axis as a rotation axis is defined as a pitch angle, and a rotation angle within the horizontal plane with the z axis as a rotation axis is defined as a yaw angle.

As illustrated in FIG. 2, the feature quantity data generation unit 12 (also referred to as a feature quantity data generation device) includes an acquisition unit 121, a normalization unit 122, an extraction unit 123, a generation unit 125, and a feature quantity data output unit 127. For example, the feature quantity data generation unit 12 is achieved by a microcomputer or a microcontroller that performs overall control and data processing of the gait measuring device 10. For example, the feature quantity data generation unit 12 includes a central processing unit (CPU), a random access memory (RAM), a read only memory (ROM), a flash memory, and the like. The feature quantity data generation unit 12 controls the acceleration sensor 111 and the angular velocity sensor 112 to measure the angular velocity and the acceleration. For example, the feature quantity data generation unit 12 may be implemented on a mobile terminal (not illustrated) side carried by a subject (user).

The acquisition unit 121 acquires accelerations in three axes directions from the acceleration sensor 111. In addition, the acquisition unit 121 acquires angular velocities around three axes from the angular velocity sensor 112. For example, the acquisition unit 121 performs analog-to-digital conversion (AD conversion) on the acquired physical quantities (analog data) such as angular velocity and acceleration. The physical quantity (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in three axes directions. The angular velocity data includes angular velocity vectors around three axes. The acceleration data and the angular velocity data are associated with acquisition times of the data. In addition, the acquisition unit 121 may add correction such as a mounting error, temperature correction, and linearity correction to the acceleration data and the angular velocity data.

The normalization unit 122 acquires sensor data from the acquisition unit 121. The normalization unit 122 extracts time-series data (also referred to as walking waveform data) for one gait cycle from the time-series data of the acceleration in the three axes directions and the angular velocity around the three axes included in the sensor data. The normalization unit 122 normalizes (also referred to as first normalization) the time of the extracted walking waveform data for one gait cycle to a gait cycle of 0 to 100% (percent). Timing such as 1% or 10% included in the gait cycle of 0 to 100% is also referred to as a walking phase. Furthermore, the normalization unit 122 normalizes (also referred to as second normalization) the first normalized walking waveform data for one gait cycle in such a way that the stance phase becomes 60% and the swing phase becomes 40%. The stance phase is a period in which at least a part of the back side of the foot is in contact with the ground. The swing phase is a period in which the back side of the foot is separated from the ground. When the walking waveform data is subjected to the second normalization, deviation of the walking phase from which the feature quantity is extracted can be suppressed from fluctuating due to the influence of disturbance.

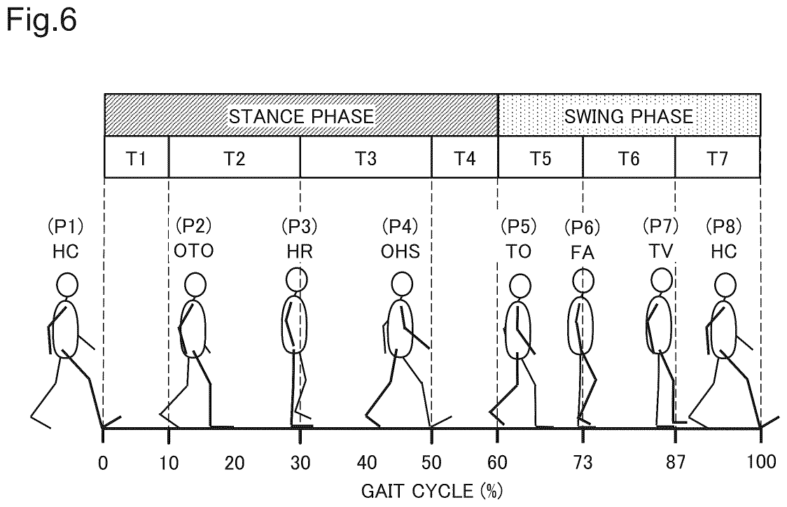

FIG. 6 is a conceptual diagram for explaining one gait cycle with the right foot as a reference. One gait cycle with the left foot as a reference is also similar to that of the right foot. The horizontal axis of FIG. 6 is one gait cycle of the right foot with a time point at which the heel of the right foot lands on the ground as a starting point and a time point at which the heel of the right foot lands on the ground the next time as an ending point. In the horizontal axis in FIG. 6, first normalization is performed with one gait cycle as 100%. Furthermore, in the horizontal axis of FIG. 6, the second normalization is performed such that the stance phase becomes 60% and the swing phase becomes 40%. The one gait cycle of one foot is roughly divided into a stance phase in which at least a part of the back side of the foot is in contact with the ground and a swing phase in which the back side of the foot is separated from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, in walking, a plurality of events (also referred to as walking events) occur. P1 represents an event (heel-ground contact) in which the heel of the right foot touches the ground (HC: Heel-Contact). P2 represents an event (toe of opposite foot ground separation) in which the toe of the left foot separates from the ground while the sole of the right foot is in contact with the ground (OTO: Opposite-Toe-Off). P3 represents an event (heel lifts up) in which the heel of the right foot lifts up while the sole of the right foot in contact with the ground (HR: Heel Rise). P4 is an event (heel of opposite foot ground contact) in which the heel of the left foot touches the ground (OHS: Opposite Heel Strike). P5 represents an event (toe-ground separation) in which the toe of the right foot separates from the ground while the sole of the left foot is in contact with the ground (TO: Toe-Off). P6 represents an event (foot crossing) in which the left foot and the right foot cross each other while the sole of the left foot is in contact with the ground (FA: Foot Adjacent). P7 represents an event (tibia vertical) in which the tibia of the right foot is substantially perpendicular to the ground while the sole of the left foot is in contact with the ground (TV: Tibia Vertical). P8 represents an event (heel-ground contact) in which the heel of the right foot touches the ground (HC: Heel-Contact). P8 corresponds to the ending point of the gait cycle starting from P1 and also corresponds to the start point of the next gait cycle. FIG. 6 is an example, and does not limit events that occur during walking or names of these events.

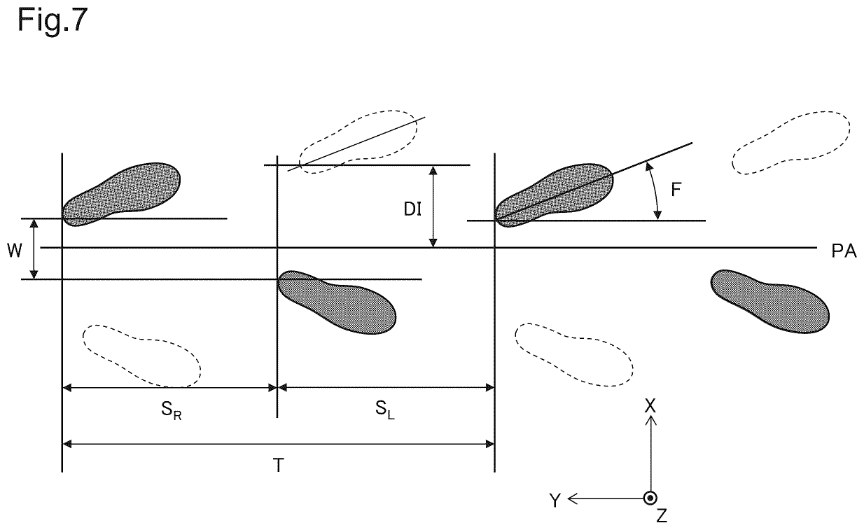

FIG. 7 is a conceptual diagram for describing an example of a gait parameter. FIG. 7 illustrates a right foot step length SR, a left foot step length SL, a stride length T, a step size W, a foot angle F, and a circumduction amount D1. In addition, FIG. 7 illustrates an advancing axis PA which is parallel to the axis (Y axis) in the advancing direction and corresponds to a trajectory connecting the middle of the left and right feet. The right foot step length SR is a difference in the Y coordinate between the heel of the right foot and the heel of the left foot when the state in which the sole of the left foot is in contact with the ground transitions to the state in which the heel of the right foot swung out in the advancing direction lands. The left foot step length SL is a difference in the Y coordinate between the heel of the left foot and the heel of the right foot when the state in which the sole of the right foot is in contact with the ground transitions to the state in which the heel of the left foot swung out in the advancing direction lands. The stride length T is the sum of the right foot step length SR and the left foot step length SL. The step size W is an interval between the right foot and the left foot. In FIG. 7, the step size W is a difference between the center line (X coordinate) of the heel of the right foot in the ground contacting state and the center line (X coordinate) of the heel of the left foot in the ground contacting state. The foot angle F is an angle formed by the center line of the foot and the advancing direction (Y axis) in a state where the sole surface is in contact with the ground. In the present example embodiment, the foot angle in a state where the foot is in contact with the ground is evaluated in the stance phase. The circumduction amount D1 is a distance between the advancing axis PA and the foot at a timing when the central axis of the foot is farthest from the advancing axis PA in the swing phase. In the present example embodiment, the circumduction amount D1 is normalized by the height due to the influence of the length of the lower limb.

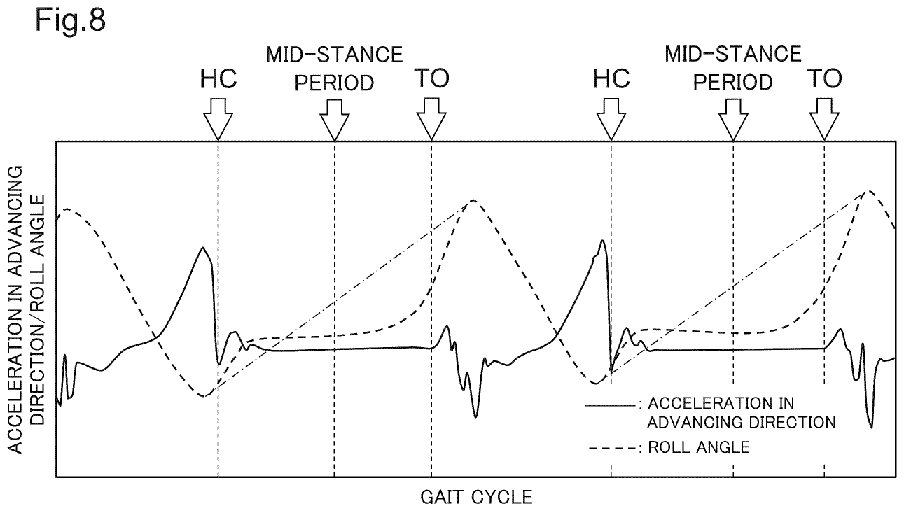

FIG. 8 is a diagram for describing an example of detecting the heel-ground contact HC and the toe-ground separation TO from the time-series data (solid line) of the acceleration in the advancing direction (acceleration in the Y direction). The timing of the heel-ground contact HC is the timing of the minimum peak immediately after the maximum peak appearing in the time-series data of the acceleration in the advancing direction (acceleration in the Y direction). The maximum peak serving as a mark of the timing of the heel-ground contact HC corresponds to the maximum peak of the walking waveform data for one gait cycle. A section between the consecutive heel-ground contacts HC is one gait cycle. The timing of the toe-ground separation TO is the timing of rise of the maximum peak appearing after the period of the stance phase in which the fluctuation does not appear in the time-series data of the acceleration in the advancing direction (acceleration in the Y direction). FIG. 8 also illustrates time-series data (broken line) of the roll angle (angular velocity around the X axis). The timing at the midpoint between the timing at which the roll angle is minimum and the timing at which the roll angle is maximum corresponds to the mid-stance period. For example, parameters (also referred to as gait parameters) such as walking speed, step size, circumduction, internal rotation/external rotation, and plantar flexion/dorsal flexion can be obtained with reference to the mid-stance period.

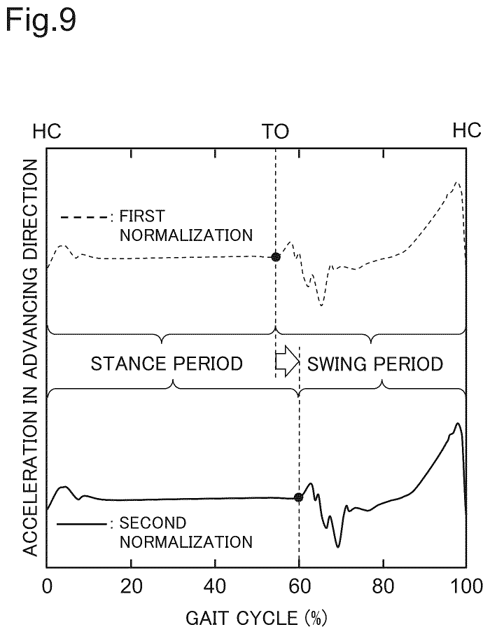

FIG. 9 is a diagram for explaining an example of the walking waveform data normalized by the normalization unit 122. The normalization unit 122 detects the heel-ground contact HC and the toe-ground separation TO from the time-series data of the acceleration in the advancing direction (acceleration in the Y direction). The normalization unit 122 extracts a section between consecutive heel-ground contacts HC as walking waveform data for one gait cycle. The normalization unit 122 converts the horizontal axis (time axis) of the walking waveform data for one gait cycle into a gait cycle of 0 to 100% by the first normalization. In FIG. 9, the walking waveform data after the first normalization is indicated by a broken line. In the walking waveform data (broken line) after the first normalization, the timing of the toe-ground separation TO is deviated from 60%.

In the example of FIG. 9, the normalization unit 122 normalizes a section from the heel-ground contact HC in which the walking phase is 0% to the toe-ground separation TO subsequent to the heel-ground contact HC to 0 to 60%. In addition, the normalization unit 122 normalizes a section from the toe-ground separation TO to the heel-ground contact HC in which the walking phase is 100% subsequent to the toe-ground separation TO to 60 to 100%. As a result, the walking waveform data for one gait cycle is normalized to a section (stance phase) in which the gait cycle is 0 to 60% and a section (swing phase) in which the gait cycle is 60 to 100%. In FIG. 9, the walking waveform data after the second normalization is indicated by a solid line. In the walking waveform data (solid line) after the second normalization, the timing of the toe-ground separation TO coincides with 60%.

FIGS. 8 to 9 illustrate examples in which the walking waveform data for one gait cycle is extracted/normalized based on the acceleration in the advancing direction (acceleration in the Y direction). With respect to acceleration/angular velocity other than the acceleration in the advancing direction (acceleration in the Y direction), the normalization unit 122 extracts/normalizes the walking waveform data for one gait cycle in accordance with the gait cycle of the acceleration in the advancing direction (acceleration in the Y direction). Furthermore, the normalization unit 122 may generate time-series data of angles around three axes by integrating time-series data of angular velocities around the three axes. In this case, the normalization unit 122 also extracts/normalizes the walking waveform data for one gait cycle in accordance with the gait cycle of the acceleration in the advancing direction (acceleration in the Y direction) with respect to the angle around the three axes.

The normalization unit 122 may extract/normalize the walking waveform data for one gait cycle based on acceleration/angular velocity other than the acceleration in the advancing direction (acceleration in the Y direction) (not illustrated). For example, the normalization unit 122 may detect the heel-ground contact HC and the toe-ground separation TO from the time-series data of the acceleration in the perpendicular direction (acceleration in the Z direction. The timing of the heel-ground contact HC is a timing of a steep minimum peak appearing in the time-series data of the acceleration in the perpendicular direction (acceleration in the Z direction). At the timing of the steep minimum peak, the value of the acceleration in the perpendicular direction (acceleration in the Z direction) becomes substantially zero. The minimum peak serving as a mark of the timing of the heel-ground contact HC corresponds to the minimum peak of the walking waveform data for one gait cycle. A section between the consecutive heel-ground contacts HC is one gait cycle. The timing of the toe-ground separation TO is a timing of an inflection point in the middle at which the time-series data of the acceleration in the perpendicular direction (acceleration in the Z direction) gradually increases after passing through a section of small fluctuation after the maximum peak immediately after the heel-ground contact HC. Furthermore, the normalization unit 122 may extract/normalize the walking waveform data for one gait cycle based on both the acceleration in the advancing direction (acceleration in the Y direction) and the acceleration in the perpendicular direction (acceleration in the Z direction). Furthermore, the normalization unit 122 may extract/normalize the walking waveform data for one gait cycle based on acceleration, angular velocity, angle, and the like other than the acceleration in the advancing direction (acceleration in the Y direction) and the acceleration in the perpendicular direction (acceleration in the Z direction).

The extraction unit 123 acquires walking waveform data for one gait cycle normalized by the normalization unit 122. The extraction unit 123 extracts a feature quantity used for estimation of muscle strength from the walking waveform data for one gait cycle. The extraction unit 123 extracts a feature quantity for each walking phase cluster from a walking phase cluster obtained by integrating temporally continuous walking phases based on a preset condition. The walking phase cluster includes at least one walking phase. The walking phase cluster also includes a single walking phase. The walking waveform data and the walking phase from which the feature quantity used for estimation of the muscle strength is extracted will be described later.

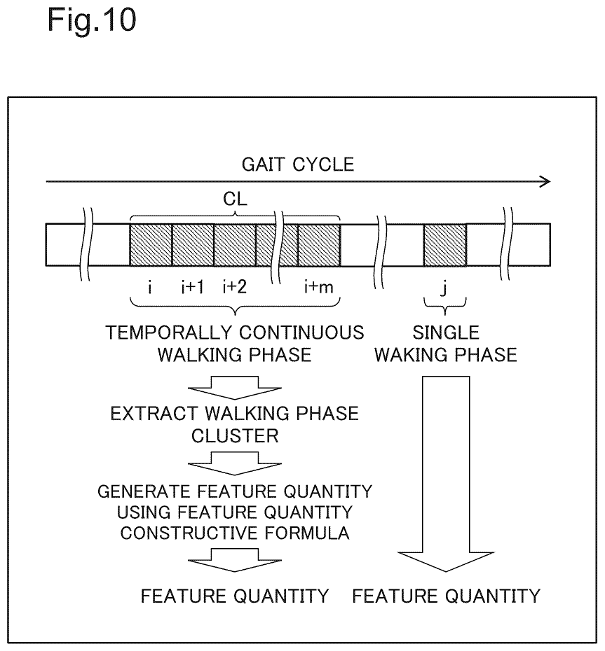

FIG. 10 is a conceptual diagram for describing extraction of a feature quantity for estimating muscle strength from walking waveform data for one gait cycle. For example, the extraction unit 123 extracts temporally continuous walking phases i to i+m as the walking phase cluster CL (i and m are natural numbers.). The walking phase cluster CL includes m walking phases (components). That is, the number of walking phases (components) (also referred to as the number of components) constituting the walking phase cluster CL is m. FIG. 10 illustrates an example in which the walking phase has an integer value, but the walking phase may be subdivided into after the decimal point. When the walking phase is subdivided into after the decimal point, the number of components of the walking phase cluster CL becomes the number corresponding to the number of data points in the section of the walking phase cluster. The extraction unit 123 extracts a feature quantity from each of the walking phases i to i+m. In a case where the walking phase cluster CL includes a single walking phase j, the extraction unit 123 extracts a feature quantity from the single walking phase j (j is a natural number).

The generation unit 125 generates the feature quantity (second feature quantity) of the walking phase cluster by applying the feature quantity constructive formula to the feature quantity (first feature quantity) extracted from each of the walking phases constituting the walking phase cluster. The feature quantity constructive formula is a preset calculation formula for generating the feature quantity of the walking phase cluster. For example, the feature quantity constructive formula is a calculation formula related to four arithmetic operations. For example, the second feature quantity calculated using the feature quantity constructive formula is an integral average value, an arithmetic average value, an inclination, a variation, or the like of the first feature quantity in each walking phase included in the walking phase cluster. For example, the generation unit 125 applies a calculation formula for calculating the inclination and variation of the first feature quantity extracted from each of the walking phases constituting the walking phase cluster as the feature quantity constructive formula. For example, in a case where the walking phase cluster is configured by a single walking phase, it is not possible to calculate the inclination or variation, and thus a feature quantity constructive formula for calculating an integral average value, an arithmetic average value, or the like may be used.

The feature quantity data output unit 127 outputs the feature quantity data for each walking phase cluster generated by the generation unit 125. The feature quantity data output unit 127 outputs the generated feature quantity data of the walking phase cluster to the muscle strength evaluation device 13 that uses the feature quantity data.

### [Muscle Strength Evaluation Device]

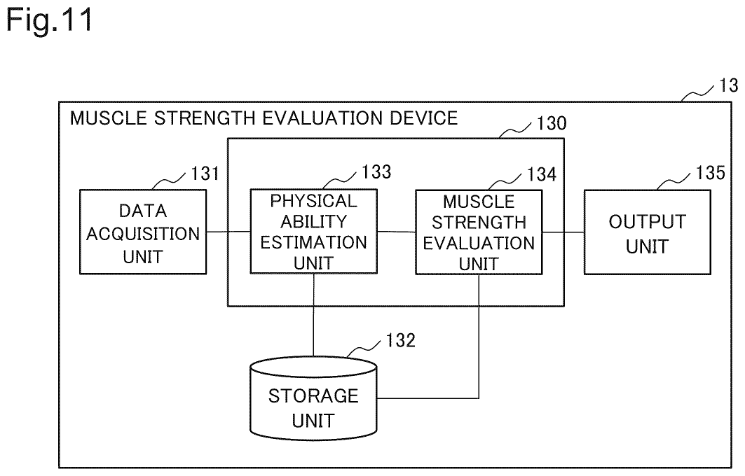

FIG. 11 is a block diagram showing an example of a configuration of the muscle strength evaluation device 13. The muscle strength evaluation device 13 includes a data acquisition unit 131, a storage unit 132, a physical ability estimation unit 133, a muscle strength evaluation unit 134, and an output unit 135. The physical ability estimation unit 133 and the muscle strength evaluation unit 134 configure an evaluation unit 130.

The data acquisition unit 131 acquires feature quantity data from the gait measuring device 10. The data acquisition unit 131 outputs the received feature quantity data to the physical ability estimation unit 133. The data acquisition unit 131 may receive the feature quantity data from the gait measuring device 10 via a wire such as a cable, or may receive the feature quantity data from the gait measuring device 10 via wireless communication. For example, the data acquisition unit 131 is configured to receive the feature quantity data from the gait measuring device 10 via a wireless communication function (not illustrated) conforming to a standard such as Bluetooth (registered trademark) or WiFi (registered trademark). The communication function of the data acquisition unit 131 may conform to a standard other than Bluetooth (registered trademark) or WiFi (registered trademark).

The storage unit 132 stores an estimation model for estimating the muscle strength of the evaluation target muscle using the feature quantity data extracted from the walking waveform data. The storage unit 132 stores an estimation model for estimating the muscle strength of the evaluation target muscle machine learned for a plurality of subjects. For example, the storage unit 132 stores an estimation model that outputs a muscle strength index (also referred to as a muscle strength score) of the evaluation target muscle according to the input of the feature quantity data extracted from the walking waveform data.

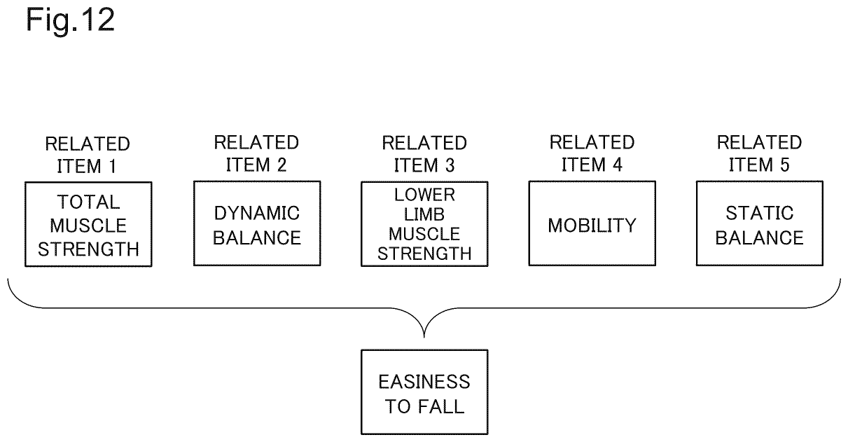

FIG. 12 is a conceptual diagram for explaining five related items (also referred to as five items) related to the muscle strength of the evaluation target muscle. The five items of total muscle strength (grip strength) of the whole body, dynamic balance, lower limb muscle strength, mobility, and static balance are related to the muscle strength of the evaluation target muscle. The details on the five items of total muscle strength (grip strength) of the whole body, dynamic balance, lower limb muscle strength, mobility, and static balance related to the muscle strength of the evaluation target muscle will be described later.

For example, the storage unit 132 stores an estimation model that outputs muscle strength index (muscle strength score) of the muscle to be evaluated according to an input of feature quantity data common to estimation of the muscle strength of the evaluation target muscle. For example, the storage unit 132 stores an estimation model (also referred to as a physical ability estimation model) that outputs the score of each of the five items according to the input of the feature quantity data used to estimate the score of each of the five items. For example, the storage unit 132 stores an estimation model (also referred to as a muscle strength estimation model) that outputs a muscle strength index (muscle strength score) of the evaluation target muscle according to the input of the scores of the five items.

The estimation model may be stored in the storage unit 132 at the timing such as at the time of factory shipment of a product, calibration before the user uses the muscle strength evaluation system 1, or the like. For example, an estimation model saved in a storage device such as an external server may be used. In that case, the estimation model may be configured to be used via an interface (not illustrated) connected to the storage device.

The physical ability estimation unit 133 acquires the feature quantity data from the data acquisition unit 131. The physical ability estimation unit 133 estimates the physical ability of the user by using the acquired feature quantity data. The physical ability estimation unit 133 inputs the feature quantity data to the physical ability estimation model stored in the storage unit 132. The physical ability estimation unit 133 estimates the physical ability of the user by using the physical ability index (score) corresponding to the physical ability output from the physical ability estimation model. For example, the physical ability estimation unit 133 estimates the physical ability of the user by using the score of each of the five items according to the input of the feature quantity data used to estimate the score of each of the five items. The physical ability estimation unit 133 outputs an estimation result using the physical ability index (score) output from the physical ability estimation model to the muscle strength evaluation unit 134. For example, the physical ability estimation unit 133 outputs the physical ability index (score) output from the physical ability estimation model according to the input of the feature quantity data to the muscle strength evaluation unit 134.

The muscle strength evaluation unit 134 acquires, from the physical ability estimation unit 133, an estimation result of the physical ability estimated by the physical ability estimation unit 133 using the feature quantity data. The muscle strength evaluation unit 134 estimates the muscle strength of the evaluation target muscle by using the acquired estimation result. For example, the muscle strength evaluation unit 134 acquires a physical ability index (score) output from the physical ability estimation model according to the input of the feature quantity data. The muscle strength evaluation unit 134 inputs a physical ability index (score) to the muscle strength estimation model stored in the storage unit 132. The muscle strength evaluation unit 134 evaluates the muscle strength of the evaluation target muscle of the user by using the muscle strength index (score) of the evaluation target muscle output from the muscle strength estimation model. For example, the muscle strength evaluation unit 134 outputs the muscle strength index (score) of the evaluation target muscle output from the muscle strength estimation model to the output unit 135 according to the input of the score of each of the five items.

The physical ability estimation unit 133 and the muscle strength evaluation unit 134 may use the estimation model saved in an external storage device constructed in a cloud, a server, or the like. In this case, the physical ability estimation unit 133 and the muscle strength evaluation unit 134 are configured to use the physical ability estimation model via an interface (not illustrated) connected to the storage device.

The output unit 135 outputs the evaluation result of the muscle strength of the evaluation target muscle by the muscle strength evaluation unit 134. For example, the output unit 135 displays the evaluation result related to the muscle strength of the evaluation target muscle on a screen of a mobile terminal of a subject (user). For example, the output unit 135 outputs the evaluation result to an external system or the like that uses the evaluation result. The use of the evaluation result output from the muscle strength evaluation device 13 is not particularly limited.

For example, the muscle strength evaluation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the muscle strength evaluation device 13 is connected to a mobile terminal via a wire such as a cable. For example, the muscle strength evaluation device 13 is connected to a mobile terminal via wireless communication. For example, the muscle strength evaluation device 13 is connected to a mobile terminal via a wireless communication function (not illustrated) conforming to a standard such as Bluetooth (registered trademark) or WiFi (registered trademark). The communication function of the muscle strength evaluation device 13 may conform to a standard other than Bluetooth (registered trademark) or WiFi (registered trademark). The evaluation result of the muscle strength of the evaluation target muscle may be used by an application installed in the mobile terminal. In that case, the mobile terminal executes processing using the evaluation result by application software or the like installed in the mobile terminal.

Next, each of the five items of total muscle strength (grip strength) of the whole body, dynamic balance, lower limb muscle strength, mobility, and static balance related to the evaluation target muscle shown in FIG. 12 will be individually described. After describing each of the five items, feature quantities used for estimating the muscle strength of the evaluation target muscle will be described. The muscle strength is estimated using the feature quantity common to the five items.

### <Related Item 1>

Related item 1 relates to total muscle strength of the whole body. There is a correlation between the total muscle strength and the grip strength. The grip strength is also correlated with the knee flexibility. One of indices of total muscle strength related to related item 1 is grip strength. For example, an estimated value of the grip strength is an index of total muscle strength. For example, a score corresponding to the estimated value of the grip strength (also referred to as a total muscle strength score) is an index of the total muscle strength. The total muscle strength score is a value obtained by scoring the grip strength, which is an index of the total muscle strength, on a preset reference. The grip strength is affected by attributes such as gender, age, and height. Therefore, the total muscle strength score may be scored based on a reference for each attribute. In particular, the grip strength is affected by gender. Therefore, the total muscle strength score may be scored based on different references depending on the gender. The index of the total muscle strength is not limited to the grip strength as long as the total muscle strength can be scored.

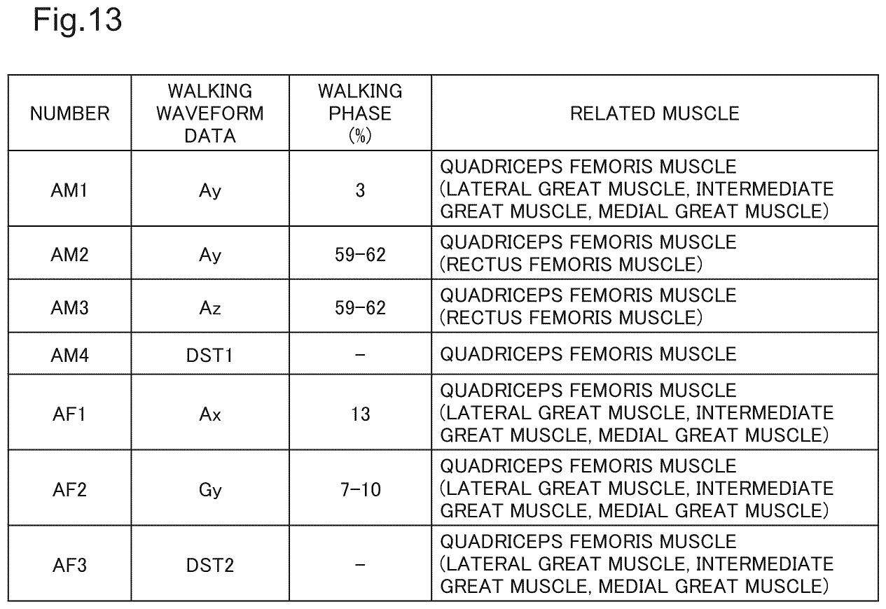

FIG. 13 is a correspondence table summarizing feature quantities used for estimation of the total muscle strength (grip strength) of the whole body. The correspondence table of FIG. 13 corresponds the number of the feature quantity, the walking waveform data from which the feature quantity is extracted, the walking phase (%) from which the walking phase cluster is extracted, and the related muscle. The walking phase from which the feature quantity used for estimation of the grip strength is extracted differs depending on the gender. For men, there is a correlation between the activity of the quadriceps femoris muscle and the grip strength. Therefore, feature quantities AM1 to AM4 extracted from the walking phase in which the features of the activity of the quadriceps femoris muscle appear are used for estimation of the grip strength of the male. For women, there is a correlation between the activities of the lateral great muscle, the intermediate great muscle, and the medial great muscle of the quadriceps femoris muscle and the grip strength. Therefore, feature quantities AF1 to AF3 extracted from the walking phase in which the features of the activities of the lateral, intermediate, and medial great muscles appear are used for estimation of the grip strength of the female.

The feature quantity AM1 is extracted from a section of the walking phase 3% of the walking waveform data Ay regarding the time-series data of the acceleration in the advancing direction (acceleration in the Y direction). The walking phase 3% is included in the load response period T1. The feature quantity AM1 mainly includes features related to the movement of the lateral, intermediate, and medial great muscles of the quadriceps femoris muscle.

The feature quantity AM2 is extracted from a section of the walking phase 59% to 62% of the walking waveform data Ay regarding the time-series data of the acceleration in the advancing direction (acceleration in the Y direction). The walking phase 59% to 62% is included in the pre-swing period T4. The feature quantity AM2 mainly includes a feature related to movement of the rectus femoris muscle of the quadriceps femoris muscle.

The feature quantity AM3 is extracted from a section of the walking phase 59% to 62% of the walking waveform data Az regarding the time-series data of the acceleration in the perpendicular direction (acceleration in the Z direction). The walking phase 59% to 62% is included in the pre-swing period T4. The feature quantity AM3 mainly includes a feature related to movement of the rectus femoris muscle of the quadriceps femoris muscle.

The feature quantity AM4 is a proportion (DST1) of a period from when the heel is in contact with the ground until the toe of the opposite foot separates from the ground in a period in which both feet are simultaneously in contact with the ground (Double Support Time (DST)). DST1 is a proportion of a period from when the heel is in contact with the ground until the toe of the opposite foot separates from the ground in one gait cycle. The feature quantity AM4 mainly includes a feature related to the quadriceps femoris muscle.

The feature quantity AF1 is extracted from a section of the walking phase 13% of the walking waveform data Ax regarding the time-series data of the acceleration in the horizontal direction (acceleration in the X direction). The walking phase 13% is included in the mid-stance period T2. The feature quantity AF1 mainly includes features related to the movement of the lateral, intermediate, and medial great muscles of the quadriceps femoris muscle.

The feature quantity AF2 is extracted from a section of the walking phase 7 to 10% of the walking waveform data Gy regarding the time-series data of the angular velocity (pitch angular velocity) within the coronal plane (around the Y axis). The walking phase 7 to 10% is included in the load response period T1. The feature quantity AF2 mainly includes features related to movement of the lateral, intermediate, and medial great muscles.

The feature quantity AF3 is a proportion (DST2) of a period from when the heel of the opposite foot is in contact with the ground until the toe separates from the ground in a period in which both feet are simultaneously in contact with the ground (Double Support Time (DST)). DST2 is a proportion of a period from when the heel of the opposite foot is in contact with the ground until the toe separates from the ground in one gait cycle. The sum of DST1 and DST2 corresponds to a period in which both feet are simultaneously in contact with the ground in one gait cycle. The feature quantity AF3 mainly includes features related to movement of the lateral, intermediate, and medial great muscles.

### <Related Item 2>

The dynamic balance Related item 2 relates to dynamic balance. can be evaluated by the grade of the functional reach test (FRT). In the present example embodiment, the FRT grade is evaluated by the distance between the fingertips (also referred to as functional reach distance) in a state where the upper limb is moved forward as much as possible from a state where both hands are raised by 90 degrees with respect to the horizontal plane to an upright position. The functional reach distance (hereinafter, referred to as an FR distance.) is a grade value of the FRT. The grade of the FRT is higher the larger the FR distance. Related item 2 may be evaluated by other than the FRT performed with both hands. For example, related item 2 may be evaluated by grades related to the FRT performed with one hand or other variations of the FRT.

The index of the dynamic balance for related item 2 is FR distance. For example, an estimate value of the FR distance is an index of the dynamic balance. For example, a score corresponding to the estimated value of the FR distance (also referred to as a dynamic balance score) is an index of the dynamic balance. The dynamic balance score is a value obtained by scoring an FR distance, which is an index of dynamic balance, with a preset reference. The dynamic balance is affected by attributes such as height. Therefore, the dynamic balance score may be scored based on a reference for each attribute. The index of the dynamic balance is not limited to the FR distance as long as the dynamic balance can be scored.

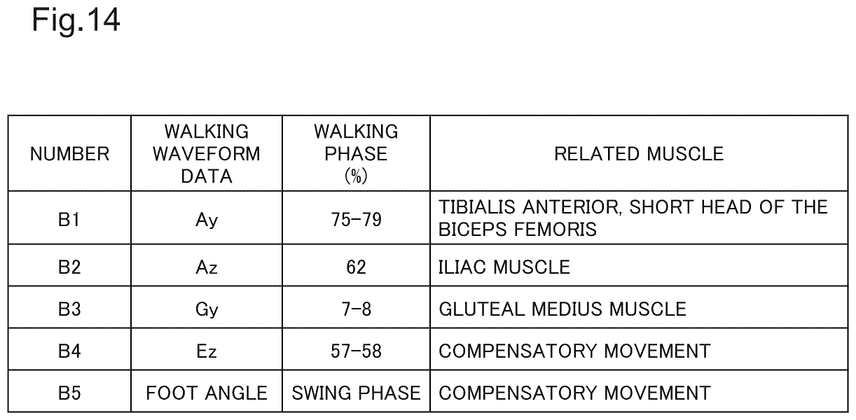

FIG. 14 is a correspondence table summarizing feature quantities used for estimation of the dynamic balance. The correspondence table of FIG. 14 corresponds the number of the feature quantity, the walking waveform data from which the feature quantity is extracted, the walking phase (%) from which the walking phase cluster is extracted, and the related muscle. The FR distance is correlated with the activities of the gluteus medius muscle, the iliac muscles, the hamstrings (the long head of the biceps femoris), the tibialis anterior, and the like, and the magnitude of the compensatory movement that makes the orientation of the part from the heel to the tip of the toe outward. Therefore, feature quantities B1 to B5 extracted from the walking phase in which these features appear are used for estimation of the FR distance.

The feature quantity B1 is extracted from a section of the walking phase 75% to 79% of the walking waveform data Ay regarding the time-series data of the acceleration in the advancing direction (acceleration in the Y direction). The walking phase 75% to 79% is included in the mid-swing period T6. The feature quantity B1 mainly includes a feature related to movement of the tibialis anterior and the short head of the biceps femoris.

The feature quantity B2 is extracted from a section of the walking phase 62% of the walking waveform data Az regarding the time-series data of the acceleration in the perpendicular direction (acceleration in the Z direction). The walking phase 62% is included in the initial swing period T5. The feature quantity B2 mainly includes a feature related to movement of the iliac muscle.

The feature quantity B3 is extracted from a section of the walking phase 7 to 8% of the walking waveform data Gy regarding the time-series data of the angular velocity within the coronal plane (around the Y axis). The walking phase 7 to 8% is included in the load response period T1. The feature quantity B3 mainly includes a feature related to movement of the gluteus medius muscle.

The feature quantity B4 is extracted from a section of the walking phase 57 to 58% of the walking waveform data Ez regarding the time-series data of the angle (posture angle) within the horizontal plane (around the Z axis). The walking phase 57% to 58% is included in the pre-swing period T4. The feature quantity B4 mainly includes a feature related to a compensatory movement. The compensatory movement is a movement of changing the foot angle to acquire stability in order to compensate for a decrease in balance ability and muscle function associated with aging.

The feature quantity B5 is an average value of the foot angles within the horizontal plane in the swing phase. For example, the feature quantity B5 is an average value in the swing phase of the walking waveform data Ez. In other words, the feature quantity B5 is an integral value of the walking waveform data Gz regarding the time-series data of the angular velocity within the horizontal plane (around the Z axis). The feature quantity B5 mainly includes a feature related to a compensatory movement.

### <Related Item 3>

Related item 3 relates to lower limb muscle strength. The lower limb muscle strength can be evaluated by a grade of a chair standing test. In the present example embodiment, the grade of the chair standing test is evaluated five times in which standing and sitting on and from a chair is repeated five times. The five-time chair standing test is also referred to as an SS-5 (Sit to Stand-5) test. The grade of the five-time chair standing test is evaluated by the time for repeating standing and sitting on and from a chair five times (also referred to as stand-sit time). The stand-sit time is a grade value of the SS-5 test. The grade of the SS-5 test is higher the shorter the stand-sit time. Evaluation may be made with the grade of the 30 second chair standing (CS-30) test in which the number of stand-sit movements to and from the chair in 30 seconds is measured.

The index of the lower limb muscle strength related to the related item 3 is a stand-sit time. For example, an estimated value of the five stand-sit time is an index of the lower limb muscle strength. For example, a score corresponding to the estimated value of the stand-sit time (also referred to as a lower limb muscle strength score) is an index of the lower limb muscle strength. The lower limb muscle strength score is a value obtained by scoring a stand-sit time, which is an index of the lower limb muscle strength, on a preset reference. The lower limb muscle strength is affected by attributes such as age. Therefore, the lower limb muscle strength score may be scored based on a reference for each attribute. The index of lower limb muscle strength is not limited to the stand-sit time as long as the lower limb muscle strength can be scored.

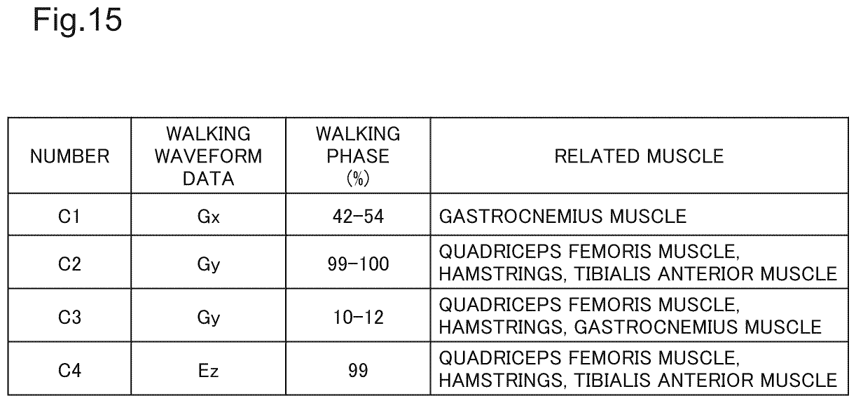

FIG. 15 is a correspondence table summarizing feature quantities used for estimation of the lower limb muscle strength. The correspondence table of FIG. 15 corresponds the number of the feature quantity, the walking waveform data from which the feature quantity is extracted, the walking phase (%) from which the walking phase cluster is extracted, and the related muscle. The stand-sit time is correlated with quadriceps femoris muscle, hamstrings, tibialis anterior muscle, and gastrocnemius muscle. Therefore, feature quantities C1 to C4 extracted from the walking phase in which these features appear are used for estimation of the stand-sit time.

The feature quantity C1 is extracted from the section of the walking phase 42 to 54% of the walking waveform data Gx related to the time-series data of the angular velocity within the sagittal plane (around the X axis). The walking phase 42 to 54% is a section from the terminal stance period T3 to the pre-swing period T4. The feature quantity C1 mainly includes a feature related to movement of the gastrocnemius muscle.

The feature quantity C2 is extracted from a section of the walking phase 99 to 100% of the walking waveform data Gy regarding the time-series data of the angular velocity within the coronal plane (around the Y axis). The walking phase 99 to 100% is the final stage of the terminal swing period T7. The feature quantity C2 mainly includes a feature related to movement of the quadriceps femoris muscle, the hamstrings, and the tibialis anterior muscle.

The feature quantity C3 is extracted from a section of the walking phase 10 to 12% of the walking waveform data Gy regarding the time-series data of the angular velocity within the coronal plane (around the Y axis). The walking phase 10 to 12% is the early stage of the mid-stance period T2. The feature quantity C3 mainly includes a feature related to movement of the quadriceps femoris muscle, the hamstrings, and the gastrocnemius muscle.

The feature quantity C4 is extracted from a section of the walking phase 99% of the walking waveform data Ez regarding the time-series data of the angle (posture angle) within the horizontal plane (around the Z axis). The walking phase 99% is the final stage of the terminal swing period T7. The feature quantity C4 mainly includes a feature related to movement of the quadriceps femoris muscle, the hamstrings, and the tibialis anterior muscle.

### <Related Item 4>

Related item 4 relates to mobility. The mobility can be evaluated by the grade of the time up and go (TUG) test. In the present example embodiment, the grade of the TUG test is evaluated by the time (also referred to as TUG required time) from standing up from a chair, walking to a mark 3 m (meters) ahead to change direction, and sitting down again on the chair. The TUG required time is a grade value of the TUG test. The record of the TUG test is higher the shorter the TUG required time. Related item 4 may be evaluated by the grade of a test related to mobility other than the TUG test.

The index of the mobility regarding the related item 4 is the TUG required time. For example, an estimated value of the TUG required time is an index of the mobility. For example, a score corresponding to the estimated value of the TUG required time (also referred to as a mobility score) is an index of the mobility. The mobility score is a value obtained by scoring the TUG required time, which is an index of the mobility, with a preset reference. The mobility is affected by attributes such as age. Therefore, the mobility score may be scored based on a reference for each attribute. The index of the mobility is not limited to the TUG required time as long as the mobility can be scored.

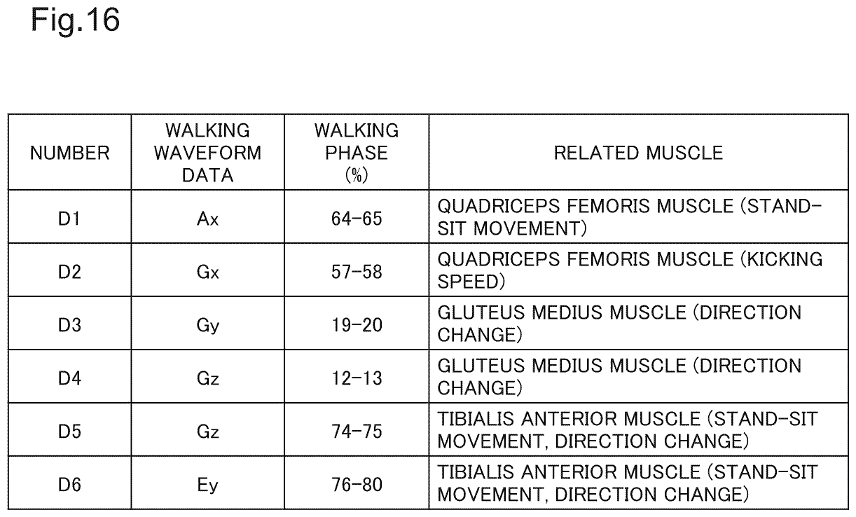

FIG. 16 is a correspondence table summarizing feature quantities used for estimation of the mobility. The correspondence table of FIG. 16 corresponds the number of the feature quantity, the walking waveform data from which the feature quantity is extracted, the walking phase (%) from which the walking phase cluster is extracted, and the related muscle. The TUG required time is correlated with the quadriceps femoris muscle, the gluteus medius muscle, and the tibialis anterior muscle. Therefore, the feature quantities D1 to D6 extracted from the walking phase in which these features appear are used for estimation of the TUG required time. The features of the tensor fasciae latae muscle appear in the walking phases 0 to 45%, and 85 to 100%. The features of the gluteus medius muscle appear in the walking phases 0 to 25%. The features of the tibialis anterior muscle appear in the walking phases 0 to 10%, and 57 to 100%.

The feature quantity D1 is extracted from a section of the walking phase 64 to 65% of the walking waveform data Ax regarding the time-series data of the acceleration in the horizontal direction (acceleration in the X direction). The walking phase 64 to 65% is included in the initial swing period T5. The feature quantity D1 mainly includes a feature related to the movement of the quadriceps femoris muscle in the stand-sit movement.

The feature quantity D2 is extracted from the section of the walking phase 57 to 58% of the walking waveform data Gx related to the time-series data of the angular velocity within the sagittal plane (around the X axis). The walking phase 57% to 58% is included in the pre-swing period T4. The feature quantity D2 mainly includes a feature related to the movement of the quadriceps femoris muscle associated with the leg kicking speed.

The feature quantity D3 is extracted from a section of the walking phase 19 to 20% of the walking waveform data Gy regarding the time-series data of the angular velocity within the coronal plane (around the Y axis). The walking phase 19 to 20% is included in the mid-stance period T2. The feature quantity D3 mainly includes a feature related to the movement of the gluteus medius muscle in the direction change.

The feature quantity D4 is extracted from a section of the walking phase 12 to 13% of the walking waveform data Ez regarding the time-series data of the angular velocity within the horizontal plane (around the Z axis). The walking phase 12 to 13% is the early stage of the mid-stance period T2. The feature quantity D4 mainly includes a feature related to the movement of the gluteus medius muscle in the direction change.

The feature quantity D5 is extracted from a section of the walking phase 74 to 75% of the walking waveform data Ez regarding the time-series data of the angular velocity within the horizontal plane (around the Z axis). The walking phase 74 to 75% is the early stage of the mid-swing period T6. The feature quantity D5 mainly includes a feature related to the movement of the tibialis anterior muscle in the stand-sit movement and the direction change.

The feature quantity D6 is extracted from a section of the walking phase 76 to 80% of the walking waveform data Ey regarding the time-series data of the angle (posture angle) within the coronal plane (around the Y axis). The walking phase 76 to 80% is included in the mid-swing period T6. The feature quantity D6 mainly includes a feature related to the movement of the tibialis anterior muscle in the stand-sit movement and the direction change.

### <Related Item 5>

Related item 5 relates to static balance. The static balance can be evaluated by the grade of a one-leg standing test. In the present example embodiment, the grade of the one-leg standing test is evaluated by a time (also referred to as a one-leg standing time) in which the eyes are closed and one leg is kept raised from the ground by 5 cm (centimeter). The one-leg standing time is a grade value of the static balance. The grade of the static balance is higher the larger the one-leg standing time. The related item 5 may be evaluated by a grade other than the eyes-closed one-leg standing test. For example, the related item 5 may be evaluated by a one-leg standing test with the eyes opened (eyes-opened one-leg standing test) or other variations of the one-leg standing test.

The index of the static balance related to the related item 5 is a one-leg standing time. For example, the estimated value of the one-leg standing time is an index of the static balance. For example, a score corresponding to the estimated value of the one-leg standing time (also referred to as a static balance score) is an index of the static balance. The static balance score is a value obtained by scoring a one-leg standing time which is an index of the static balance with a preset reference. The static balance is affected by attributes such as age and height. Therefore, the static balance score may be scored based on a reference for each attribute. The index of the static balance is not limited to the one-leg standing time as long as the static balance can be scored.

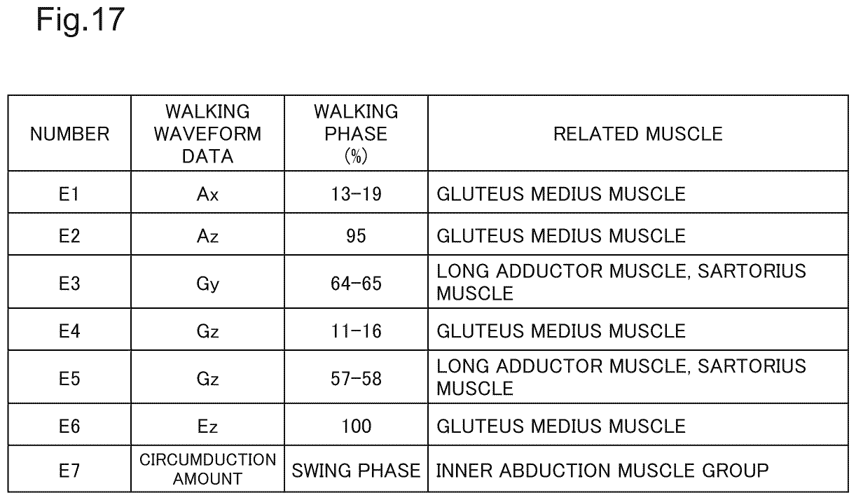

FIG. 17 is a correspondence table summarizing feature quantities used for estimation of the static balance. The correspondence table of FIG. 17 corresponds the number of the feature quantity, the walking waveform data from which the feature quantity is extracted, the walking phase (%) from which the walking phase cluster is extracted, and the related muscle. The one-leg standing time is correlated with the gluteus medius muscle, the long adductor muscle, the sartorius muscle, and the inner abduction muscle group. Therefore, the feature quantities E1 to E7 extracted from the walking phase in which these features appear are used for estimation of the one-leg standing time.

The feature quantity E1 is extracted from a section of the walking phase 13 to 19% of the walking waveform data Ax regarding the time-series data of the acceleration in the horizontal direction (acceleration in the X direction). The walking phase 13 to 19% is included in the mid-stance period T2. The feature quantity E1 mainly includes a feature related to movement of the gluteus medius muscle.

The feature quantity E2 is extracted from a section of the walking phase 95% of the walking waveform data Az regarding the time-series data of the acceleration in the perpendicular direction (acceleration in the Z direction). The walking phase 95% is the final stage of the terminal swing period T7. The feature quantity E2 mainly includes a feature related to movement of the gluteus medius muscle.

The feature quantity E3 is extracted from a section of the walking phase 64 to 65% of the walking waveform data Gy regarding the time-series data of the angular velocity within the coronal plane (around the Y axis). The walking phase 64 to 65% is included in the initial swing period T5. The feature quantity E3 mainly includes a feature related to movement of the long adductor muscle and the sartorius muscle.

The feature quantity E4 is extracted from a section of the walking phase 11 to 16% of the walking waveform data Gz regarding the time-series data of the angular velocity within the horizontal plane (around the Z axis). The walking phase 11 to 16% is included in the mid-stance period T2. The feature quantity E4 mainly includes a feature related to movement of the gluteus medius muscle.

The feature quantity E5 is extracted from a section of the walking phase 57 to 58% of the walking waveform data Gz regarding the time-series data of the angular velocity within the horizontal plane (around the Z axis). The walking phase 57 to 58% is included in the pre-swing period T4. The feature quantity E5 mainly includes a feature related to movement of the long adductor muscle and the sartorius muscle.

The feature quantity E6 is extracted from a section of the walking phase 100% of the walking waveform data Ez regarding the time-series data of the angle (posture angle) within the horizontal plane (around the Z axis). The walking phase 100% corresponds to the timing of heel-ground contact at which the terminal swing period T7 switches to the load response period T1. The feature quantity of the walking waveform data Ez in the walking phase 100% corresponds to a foot angle in a state where the sole is in contact with the ground. The feature quantity E6 mainly includes a feature related to the movement of the gluteus medius muscle.

The feature quantity E7 is a distance (circumduction amount) between the advancing axis and the foot at a timing when the central axis of the foot is farthest from the advancing axis in the swing phase. The feature quantity E7 is a circumduction amount normalized by the height of the subject. The feature quantity E7 mainly includes a feature related to the movement of the inner abduction muscle.

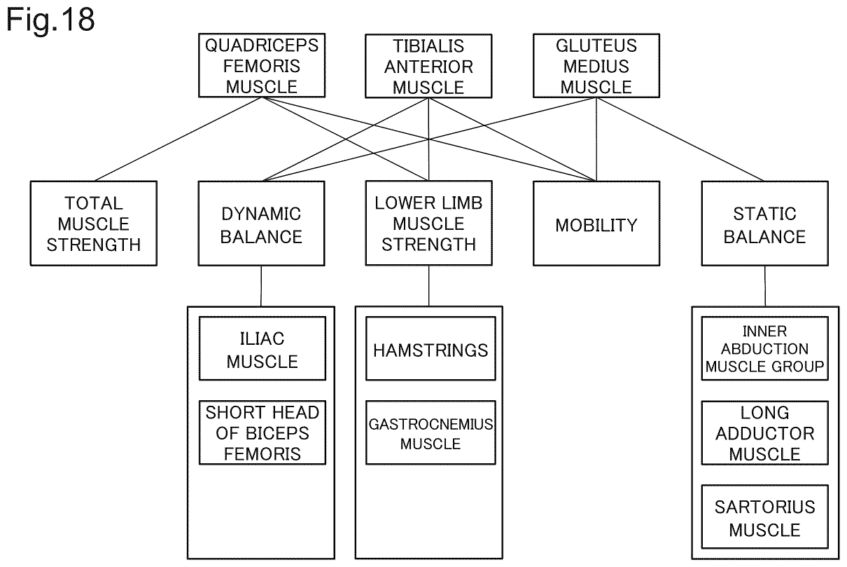

FIG. 18 is a diagram summarizing an example of a relationship between five items related to the risk of falling and muscles. In FIG. 18, five items related to the risk of falling and muscles are connected by a solid line. A solid line illustrated in FIG. 18 indicates a representative relationship. It does not indicate that there is no correlation between the item and the muscle where the solid line is not drawn. In addition, FIG. 18 includes a generic name of several muscles instead of a single muscle. The muscles shown in FIG. 18 are examples of the evaluation target muscles related to the risk of falling. The muscles shown in FIG. 18 are not all the evaluation target muscles. The muscle strength evaluation unit 134 evaluates the activities of the muscles related to the five items according to the relationship of indices (scores) of the five items related to the risk of falling.

The quadriceps femoris muscle, the tibialis anterior muscle, and the gluteus medius muscle are associated with a plurality of items. The quadriceps femoris muscle is associated with total muscle strength, lower limb muscle strength, and mobility. The quadriceps femoris muscle is associated with dynamic balance, lower limb muscle strength, and mobility. The gluteus medius muscle is associated with dynamic balance, mobility, and static balance.

For example, if the scores of the total muscle strength, the lower limb muscle strength, and the mobility are smaller than the reference values, the muscle strength evaluation unit 134 evaluates that the muscle strength of the quadriceps femoris muscle is lowering. For example, if the scores of the dynamic balance, the lower limb muscle strength, and the mobility are smaller than the reference values, the muscle strength evaluation unit 134 evaluates that the muscle strength of the tibialis anterior muscle is lowering. For example, if the scores of the dynamic balance, the mobility, and the static balance are smaller than the reference values, the muscle strength evaluation unit 134 evaluates that the muscle strength of the gluteus medius muscle is lowering.

The iliac muscle, the short head of the biceps femoris, the hamstrings, the gastrocnemius muscle, the inner abduction muscle group, the long adductor muscle, and the sartorius muscle are associated with a single item. The iliac muscle and the short head of the biceps femoris are associated with the dynamic balance. The hamstrings and the gastrocnemius muscle are associated with the lower limb muscle strength. The inner abduction muscle group, the long adductor muscle, and the sartorius muscle are associated with the static balance.

For example, if the score of the dynamic balance is smaller than the reference value, the muscle strength evaluation unit 134 evaluates that the muscle strengths of the iliac muscle and the short head of the biceps femoris are lowering. For example, if the score of lower limb muscle strength is smaller than the reference value, the muscle strength evaluation unit 134 evaluates that the muscle strengths of the hamstrings and the gastrocnemius muscle are lowering. For example, if the score of the static balance is smaller than the reference value, the muscle strength evaluation unit 134 evaluates that the muscle strengths of the inner abduction muscle group, the long adductor muscle, and the sartorius muscle are lowering.

The fall risk can be evaluated according to the scores of five items related to the risk of falling. For example, the fall risk R can be calculated using the following Equation 1.

$$R = A \times SA + B \times SB + C \times SC + D \times SD + E \times SE \qquad ( 1 )$$

In Equation 1 above, SA is the total muscle strength score of the whole body. SB is a dynamic balance score. SC is a lower limb muscle strength score. SD is a mobility score. SE is a static balance score. The total muscle strength score S1, the dynamic balance score S2, the lower limb muscle strength score S3, the mobility score S4, and the static balance score S5 of the whole body can be estimated by using estimation models corresponding to the respective scores.

In Equation 1 above, A to E are normalized specific gravity coefficients. The specific gravity coefficients A to E are set in advance according to known knowledge. For example, the specific gravity coefficients A to E can be determined according to an operation in a test of physical ability corresponding to each specific gravity coefficient. For example, the specific gravity coefficients A to E can be determined according to the ratio of the myoelectrical signal intensity of the evaluation target muscle in the test of physical ability corresponding to each specific gravity coefficient. For example, for a plurality of subjects, a myoelectric sensor is attached to a measurement site of the evaluation target muscle set in advance, and a test regarding five items related to the risk of falling is performed. By performing such a test, the specific gravity coefficients A to E can be set according to the ratio of the myoelectric signal intensity of the evaluation target muscle.

For example, the specific gravity coefficients A to E are determined by the specific gravity of the signal intensity of the walking waveform data related to the gait cycle. When the scores of the five items related to the fall risk are estimated from the gait signal, the activity amount of each muscle in the same gait cycle is calculated as a ratio and distributed to a coefficient.

The total muscle strength of the whole body is associated with the activity of the quadriceps femoris muscle. The specific gravity of the quadriceps femoris muscle in the total muscle strength score S1 of the whole body is MAI.

The tibialis anterior muscle, the gluteus medius muscle, the iliac muscle, and the short head of biceps femoris are associated with the dynamic balance. The specific gravity of the tibialis anterior muscle in the dynamic balance score S2 is MB2. The specific gravity of the gluteus medius muscle in the dynamic balance score S2 is MB3. The specific gravity of the iliac muscle in the dynamic balance score S2 is MB4. The specific gravity of the short head of the biceps femoris in the dynamic balance score S2 is MB5.

The quadriceps femoris muscle, the tibialis anterior muscle, the hamstrings, and the gastrocnemius muscle are associated with the lower limb muscle strength. The specific gravity of the quadriceps femoris muscle in the lower limb muscle strength score S3 is MC1. The specific gravity of the tibialis anterior muscle in the lower limb muscle strength score S3 is MC2. The specific gravity of the hamstrings in the lower limb muscle strength score S3 is MC6. The specific gravity of the gastrocnemius muscle in the lower limb muscle strength score S3 is MC7.

The quadriceps femoris muscle, the tibialis anterior muscle, and the gluteus medius muscle are associated with the mobility. The specific gravity of the quadriceps femoris muscle in the mobility score S4 is MD1. The specific gravity of the tibialis anterior muscle in the mobility score S4 is MD2. The specific gravity of the gluteus medius muscle in the mobility score S4 is MD3.

The inner abduction muscle, the long adductor muscle, and the sartorius muscle are associated with the static balance. The specific gravity of the inner abduction muscle in the static balance score S5 is MD8. The specific gravity of the long adductor muscle in the static balance score S5 is MD9. The specific gravity of the sartorius muscle in the static balance score S5 is MD10.

With respect to the physical ability related to the evaluation target muscle, the muscle strength evaluation unit 134 calculates the muscle strength score of the evaluation target muscle using the product of the specific gravity of the evaluation target muscle, the specific gravity coefficient of the evaluation target muscle, and the physical ability score related to the physical ability associated with the evaluation target muscle. Hereinafter, calculation examples of the muscle strength score by the muscle strength evaluation unit 134 will be listed.

The muscle strength score MS1 of the quadriceps femoris muscle is calculated using the following Equation 2.

$$MS 1 = MA 1 \times A \times SA + MC 1 \times C \times SC + MD 1 \times D \times SD \qquad ( 2 )$$

The muscle strength score MS2 of the tibialis anterior muscle is calculated using the following Equation 3.

$$MS 2 = MB 2 \times B \times SB + MC 2 \times C \times SC + MD 2 \times D \times SD \qquad ( 3 )$$

The muscle strength score MS3 of the gluteus medius muscle is calculated using the following Equation 4.

$$MS 3 = MB 3 \times B \times SB + MC 3 \times C \times SC + MD 4 \times D \times SD \qquad ( 4 )$$

The muscle strength score MS4 of the iliac muscle is calculated using the following Equation 5.

$$MS 4 = MB 4 \times B \times SB \qquad ( 5 )$$

The muscle strength score MS5 of the short head of the biceps femoris is calculated using the following Equation 6.

$$MS 5 = MB 5 \times B \times SB \qquad ( 6 )$$

The muscle strength score MS6 of the hamstrings is calculated using the following Equation 7.

$$MS 6 = MC 6 \times C \times SC \qquad ( 7 )$$

The muscle strength score MS7 of the gastrocnemius muscle is calculated using the following Equation 8.

$$MS 7 = MC 7 \times C \times SC \qquad ( 8 )$$

The muscle strength score MS8 of the inner abduction muscle group is calculated using the following Equation 9.

$$MS 8 = ME 8 \times E \times SE \qquad ( 9 )$$

The muscle strength score MS9 of the long adductor muscle is calculated using the following Equation 10.

$$MS 9 = ME 9 \times E \times SE \qquad ( 10 )$$

The muscle strength score MS10 of the sartorius muscle is calculated using the following Equation 11.

$$MS 10 = ME 10 \times E \times SE \qquad ( 11 )$$

The above Equations 2 to 11 are calculation equation group for calculating the muscle strength of the evaluation target muscle related to the risk of falling. The calculation equation group of the above Equations 2 to 11 is an example of a muscle strength estimation model for evaluating the muscle strength of each muscle according to the input of the scores of five items related to the risk of falling. For example, the muscle strength evaluation unit 134 estimates the muscle strength of the evaluation target muscle using the above Equations 2 to 11. The calculation equation group of the above Equations 2 to 11 is an example, and does not limit the muscle strength estimation model for estimating the muscle strength of the evaluation target muscle. In addition, the number of muscles for which muscle strength is estimated is not limited to ten as described above. The muscle strength estimation model only needs to be able to estimate the muscle strength of any one of evaluation target muscles according to the input of at least one of the scores of the five items related to the risk of falling.

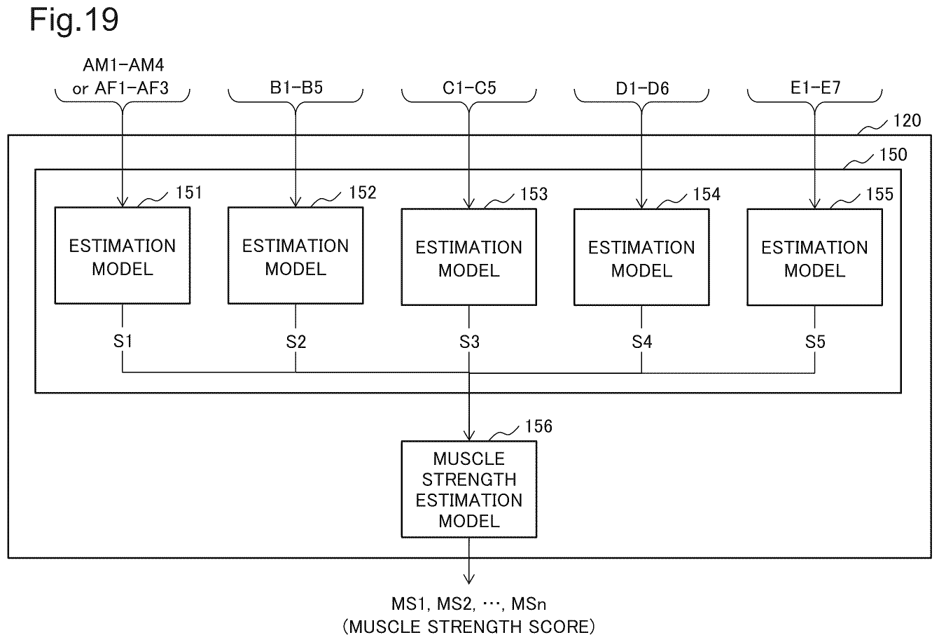

FIG. 19 is a conceptual diagram illustrating an example of the estimation model 120 that estimates the muscle strength of the evaluation target muscle using the feature quantity extracted from the walking waveform data. FIG. 19 exemplifies n evaluation target muscles (n is a natural number). The estimation model 120 includes a physical ability estimation model 150 and a muscle strength estimation model 156. The feature quantity extracted from the walking waveform data is input to the physical ability estimation model 150 that estimates scores of five items related to the fall risk. The physical ability estimation model 150 includes an estimation model 151, an estimation model 152, a muscle strength estimation model 156, an estimation model 154, and an estimation model 155. according to the input of the feature quantity extracted from the walking waveform data, the physical ability estimation model 150 outputs scores S1 to S5 of five items related to the fall risk. The scores S1 to S5 of the five items output from the physical ability estimation model 150 are input to the muscle strength estimation model 156. according to the input of the scores S1 to SS of the five items, the muscle strength estimation model 156 outputs muscle strength scores MS1 to MSn regarding the muscle strength of the evaluation target muscle.

The estimation model 151 outputs a score (total muscle strength score S1) related to the total muscle strength (grip strength) of the whole body according to the input of the feature quantities AM1 to AM4 or the feature quantities AF1 to AF3 extracted from the sensor data measured with the walking of the user. For example, the estimation model 151 may be different models for men and women. The estimation result of the estimation model 151 is not limited as long as the estimation result regarding the index of the total muscle strength is output according to the input of the feature quantity data for estimating the total muscle strength. For example, the estimation model 151 may be a model that estimates the dynamic balance using attribute data such as age and height as explanatory variables in addition to the feature quantities AM1 to AM4 or the feature quantities AF1 to AF3.

For example, the storage unit 132 stores an estimation model 151 that estimates the total muscle strength score S1 using a multiple regression prediction method. For example, the storage unit 132 stores a parameter for estimating the total muscle strength score S1 using the following Equation 12.

$$SA = m ( am 1 \times AM 1 + am 2 \times AM 2 + am 3 \times AM 3 + am 4 \times AM 4 + am 0 ) + f ( af 1 \times AF 1 + af 2 \times AF 2 + af 3 \times AF 3 + af 0 ) \qquad ( 12 )$$

In Equation 12 described above, AM1, AM2, AM3, and AM4 are feature quantities for each walking phase cluster used for estimation of the total muscle strength score S1 of the male shown in the correspondence table of FIG. 13. am1, am2, am3, and am4 are coefficients (weights) multiplied by AM1, AM2, AM3, and AM4. am0 is a constant term. AF1, AF2, and AF3 are feature quantities for each walking phase cluster used for estimation of the total muscle strength score S1 of a woman shown in the correspondence table of FIG. 13. af1, af2, and af3 are coefficients (weights) multiplied by AF1, AF2, and AF3. af0 is a constant term. m and f are flags corresponding gender. In a case where the user is a man, m is 1 and f is 0. In a case where the user is a woman, m is 0 and f is 1. For example, am0, am1, am2, am3, am4, af0, af1, af2, and af3 are stored in the storage unit 132.

The estimation model 152 outputs a score regarding the dynamic balance (dynamic balance score S2) according to the input of the feature quantities B1 to B5 extracted from the sensor data measured with the walking of the user. The estimation result of the estimation model 152 is not limited as long as the estimation result regarding the index of the dynamic balance is output according to the input of the feature quantity data for estimating the dynamic balance. For example, the estimation model 152 may be a model that estimates the dynamic balance using attribute data such as height as an explanatory variable in addition to the feature quantities B1 to B5.

For example, the storage unit 132 stores an estimation model that estimates the dynamic balance score S2 using the multiple regression prediction method. For example, the storage unit 132 stores a parameter for estimating the dynamic balance score S2 using the following Equation 13.

$$SB = b 1 \times B 1 + b 2 \times B 2 + b 3 \times B 3 + b 4 \times B 4 + b 5 \times B 5 + b 0 \qquad ( 13 )$$

In Equation 13 described above, B1, B2, B3, B4, and B5 are feature quantities for each walking phase cluster used for estimation of the dynamic balance shown in the correspondence table of FIG. 14. b1, b2, b3, b4, and b5 are coefficients (weights) multiplied by B1, B2, B3, B4, and B5. b0 is a constant term. For example, b0, b1, b2, b3, b4, and b5 are stored in the storage unit 132.

The muscle strength estimation model 156 outputs the score regarding the lower limb muscle strength (lower limb muscle strength score S3) according to the input of feature quantities C1 to C4 extracted from the sensor data measured with the walking of the user. The estimation result of muscle strength estimation model 156 is not limited as long as the estimation result regarding the index of lower limb muscle strength is output according to the input of the feature quantity data for estimating the lower limb muscle strength. For example, the muscle strength estimation model 156 may be a model that estimates dynamic balance using attribute data such as age as an explanatory variable in addition to the feature quantities C1 to C4.

For example, the storage unit 132 stores an estimation model that estimates the lower limb muscle strength score S3 using a multiple regression prediction method. For example, the storage unit 132 stores a parameter for estimating the lower limb muscle strength score S3 using the following Equation 14.

$$SC = c 1 \times C 1 + c 2 \times C 2 + c 3 \times C 3 + c 4 \times C 4 + c 0 \qquad ( 14 )$$

In Equation 14 described above, C1, C2, C3, and C4 are feature quantities for each walking phase cluster used for estimation of the lower limb muscle strength shown in the correspondence table of FIG. 15. c1, c2, c3, and c4 are coefficients (weights) multiplied by C1, C2, C3, and C4. c0 is a constant term. For example, c0, c1, c2, c3, and c4 are stored in the storage unit 132.

The estimation model 154 outputs a score regarding the mobility (mobility score S4) according to the input of the feature quantities D1 to D6 extracted from the sensor data measured with the walking of the user. The estimation result of the estimation model 154 is not limited as long as the estimation result regarding the index of the mobility is output according to the input of the feature quantity data for estimating the mobility. For example, the estimation model 154 may be a model that estimates the mobility using attribute data such as age as an explanatory variable in addition to the feature quantities D1 to D6.

For example, the storage unit 132 stores an estimation model that estimates the mobility score S4 using a multiple regression prediction method. For example, the storage unit 132 stores a parameter for estimating the mobility score S4 using the following Equation 15.

$$( 15 )$$

$$SD = d 1 \times D 1 + d 2 \times D 2 + d 3 \times D 3 + d 4 \times D 4 + d 5 \times D 5 + d 6 \times D 6 + d 0$$

In Equation 15 described above, D1, D2, D3, D4, D5, and D6 are feature quantities for each walking phase cluster used for estimation of the mobility shown in the correspondence table of FIG. 16. d1, d2, d3, d4, d5, and d6 are coefficients (weights) multiplied by D1, D2, D3, D4, D5, and D6. do is a constant term. For example, do, d1, d2, d3, d4, d5, and d6 are stored in the storage unit 132.

The estimation model 155 outputs a score related to the static balance (static balance score S5) according to the input of the feature quantities E1 to E7 extracted from the sensor data measured with the walking of the user. The estimation result of the estimation model 155 is not limited as long as the estimation result regarding the index of the static balance is output according to the input of the feature quantity data for estimating the static balance. For example, the estimation model 155 may be a model that estimates the static balance using attribute data such as age and height as explanatory variables in addition to the feature quantities E1 to E7.

For example, the storage unit 132 stores an estimation model for estimating the static balance using a multiple regression prediction method. For example, the storage unit 132 stores parameters for estimating the static balance using the following Equation 16.

$$One - leg standing time = e 1 \times E 1 + e 2 \times E 2 + e 3 \times E 3 + e 4 \times E 4 + e5 \times E 5 + e 6 \times E 6 + e 7 \times E 7 + e 0 \qquad ( 16 )$$

In Equation 16 described above, E1, E2, E3, E4, E5, E6, and E7 are feature quantities for each walking phase cluster used for estimation of the static balance shown in the correspondence table of FIG. 17. e1, e2, e3, e4, e5, e6, and e7 are coefficients (weights) multiplied by E1, E2, E3, E4, E5, E6, and E7. e0 is a constant term. For example, e0, e1, e2, e3, e4, e5, e6, and e7 are stored in the storage unit 132.

The muscle strength estimation model 156 outputs muscle strength scores MS1 to MSn of muscles related to falling according to the input of the score output from the physical ability estimation model 150. That is, the muscle strength estimation model 156 outputs the muscle strength scores MS1 to MSn according to the input of the scores of five items related to falling. For example, the muscle strength estimation model 156 includes a calculation equation group of Equations 2 to 11 described above. At least one of the scores output from the estimation model 151, the estimation model 152, the muscle strength estimation model 156, the estimation model 154, and the estimation model 155 is input to the muscle strength estimation model 156. That is, at least one of the scores output from the estimation model 151, the estimation model 152, the muscle strength estimation model 156, the estimation model 154, and the estimation model 155 merely needs to be input to the muscle strength estimation model 156. The muscle strength can be estimated with higher accuracy as the number of scores input to the muscle strength estimation model 156 increases. The estimation result of the muscle strength estimation model 156 is not limited as long as the estimation result regarding the muscle strength is output according to the input of the score output from the estimation model 152. For example, the muscle strength estimation model 156 may be a model that estimates the muscle strength using attribute data as an explanatory variable in addition to the score output from the estimation model 152.

### (Operation)

Next, an operation of the muscle strength evaluation system 1 will be described with reference to the drawings. Hereinafter, the gait measuring device 10 and the muscle strength evaluation device 13 included in the muscle strength evaluation system 1 will be individually described. With respect to the gait measuring device 10, an operation of the feature quantity data generation unit 12 included in the gait measuring device 10 will be described.

### [Gait Measuring Device]

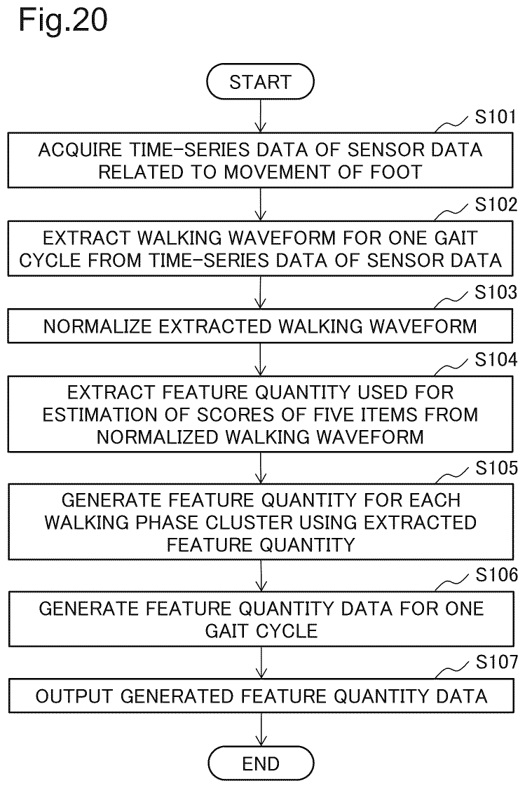

FIG. 20 is a flowchart for describing an operation of the feature quantity data generation unit 12 included in the gait measuring device 10. In the description along the flowchart of FIG. 20, the feature quantity data generation unit 12 will be described as an operation subject.

In FIG. 20, first, the feature quantity data generation unit 12 acquires time-series data of sensor data related to the movement of the foot (step S101).

Next, the feature quantity data generation unit 12 extracts walking waveform data for one gait cycle from the time-series data of the sensor data (step S102). The feature quantity data generation unit 12 detects the heel-ground contact and the toe-ground separation from the time-series data of the sensor data. The feature quantity data generation unit 12 extracts time-series data of a section between consecutive heel-ground contacts as walking waveform data for one gait cycle.

Next, the feature quantity data generation unit 12 normalizes the extracted walking waveform data for one gait cycle (step S103). The feature quantity data generation unit 12 normalizes the walking waveform data for one gait cycle to a gait cycle of 0 to 100% (first normalization). Furthermore, the feature quantity data generation unit 12 normalizes the ratio of the stance phase to the swing phase of the first normalized walking waveform data for one gait cycle to 60:40 (second normalization).

Next, the feature quantity data generation unit 12 extracts a feature quantity from the walking phase used for estimation of five items related to the fall risk with respect to the normalized walking waveform (step S104). For example, the feature quantity data generation unit 12 extracts a feature quantity input to an estimation model (first estimation model) constructed in advance.

Next, the feature quantity data generation unit 12 generates a feature quantity for each walking phase cluster by using the extracted feature quantity (step S105).

Next, the feature quantity data generation unit 12 integrates the feature quantities for each walking phase cluster to generate feature quantity data for one gait cycle (step S106).

Next, the feature quantity data generation unit 12 outputs the generated feature quantity data to the muscle strength evaluation device 13 (step S107).

[Muscle strength evaluation device]

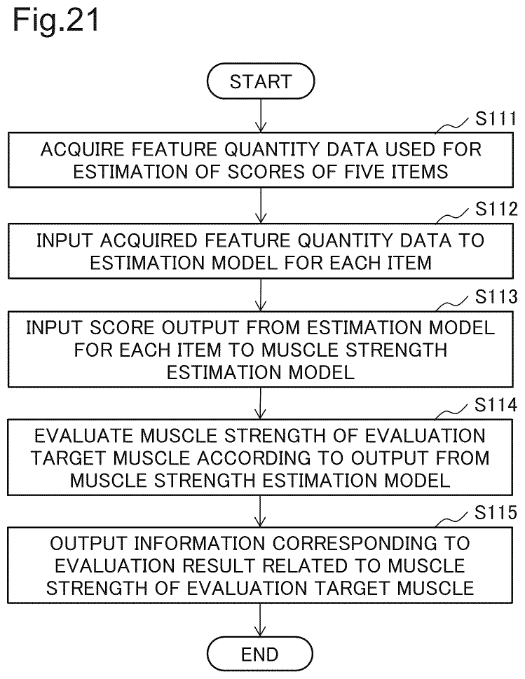

FIG. 21 is a flowchart for explaining the operation of the muscle strength evaluation device 13. In the description along the flowchart of FIG. 21, the muscle strength evaluation device 13 will be described as an operation subject.

In FIG. 21, first, the muscle strength evaluation device 13 acquires feature quantity data used for estimation of scores of five items related to the fall risk (step S111).

Next, the muscle strength evaluation device 13 inputs the acquired feature quantity data to the physical ability estimation model 150 (step S112). The feature quantity input to the physical ability estimation model 150 is input to the estimation models 151 to 155 that estimates the score of each of the five items related to the fall risk. According to the input feature quantity, the physical ability estimation model 150 outputs at least one score of five items related to the fall risk.

Next, the muscle strength evaluation device 13 inputs the scores related to the five items output from the physical ability estimation model 150 to the muscle strength estimation model 156 (step S113). According to the input score, the muscle strength estimation model 156 outputs at least one muscle strength score among the evaluation target muscles related to the fall risk.

Next, according to the output from the muscle strength estimation model 156, the muscle strength evaluation device 13 evaluates the muscle strength of the evaluation target muscle (step S114). For example, the muscle strength estimation model 156 evaluates the muscle strength of the evaluation target muscle according to the value of the score output from the muscle strength estimation model 156.

Next, the muscle strength evaluation device 13 outputs information corresponding to the evaluation result related to the muscle strength of the evaluation target muscle (step S115). For example, the evaluation result is output to a terminal device (not illustrated) carried by the user. For example, the evaluation result is output to a system that executes processes using muscle strength.

(Application example) Next, an application example according to the present example embodiment will be described with reference to the drawings. In the following application example, an example of evaluating the muscle strength of the evaluation target muscle related to the fall risk by using the feature quantity data measured by the gait measuring device 10 arranged in the shoe will be shown. For example, the function of the muscle strength evaluation device 13 is installed in a mobile terminal carried by the user.

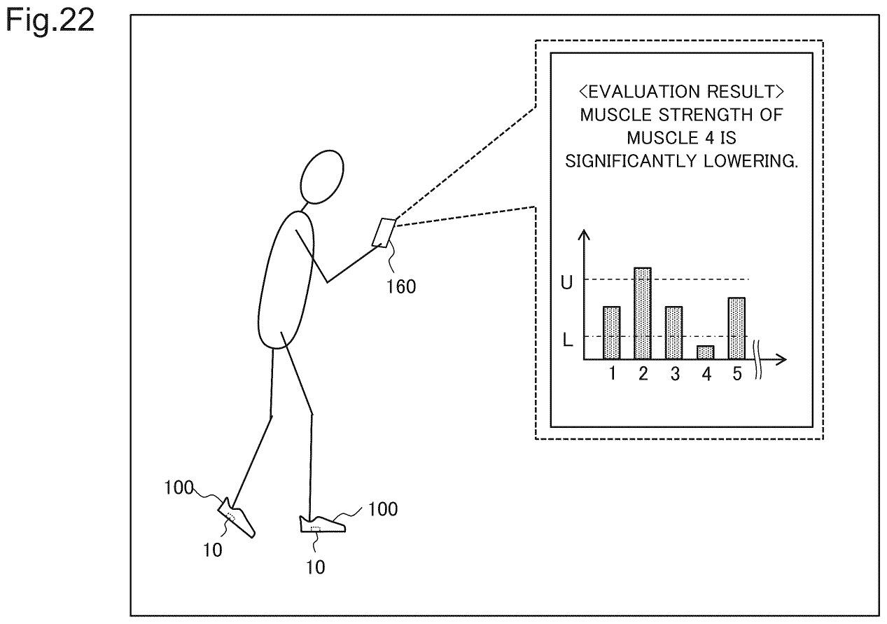

FIGS. 22 to 24 are conceptual diagrams illustrating an example in which the evaluation result by the muscle strength evaluation device 13 is displayed on the screen of the mobile terminal 160 carried by the user walking while wearing the shoes 100 on which the gait measuring device 10 is arranged. FIGS. 22 to 24 are examples in which information corresponding to the evaluation result of the muscle strength using the feature quantity data corresponding to the sensor data measured while the user is walking is displayed on the screen of the mobile terminal 160.

FIG. 22 is an example in which information corresponding to the evaluation result of the muscle strength is displayed on the screen of the mobile terminal 160. In the example of FIG. 22, an evaluation result “muscle strength of muscle 4 is significantly lowering” is displayed on the screen of the mobile terminal 160. Furthermore, in the example of FIG. 22, a graph related to the muscle strength score of the evaluation target muscle is displayed on the screen of the mobile terminal 160. In the graph of FIG. 22, the score is represented by a bar graph for each evaluation target muscle. The upper limit threshold value U and the lower limit threshold value L are illustrated in the graph of FIG. 22. The upper limit threshold value U indicates a target value of the muscle strength score. A muscle whose muscle strength score is above the upper limit threshold value U has sufficient muscle strength. The lower limit threshold value L indicates an evaluation reference value of the fall risk. A muscle whose muscle strength score is below the lower limit threshold value L may be a factor of the fall risk. In the example of FIG. 22, the muscle strength score of the muscle 4 is below the lower limit threshold value L. The user who has confirmed the evaluation result regarding the muscle strength displayed on the display unit of the mobile terminal 160 can recognize the state of his/her muscle strength by confirming the evaluation result and the graph regarding the muscle strength. In the example of FIG. 22, the score of the muscle 4 is low. The user who has confirmed the graph displayed on the display unit of the mobile terminal 160 can recognize that the muscle strength of his/her own muscle 4 is low. The information regarding the evaluation result of the muscle strength may be provided to other than the user. For example, the information regarding the evaluation result of the muscle strength may be output to a trainer that manages the physical condition of the user or a terminal device (not illustrated) used by a family of the user or the like. For example, the information regarding the evaluation result of the muscle strength may be recorded in a database (not illustrated) constructed for the purpose of health management or the like.

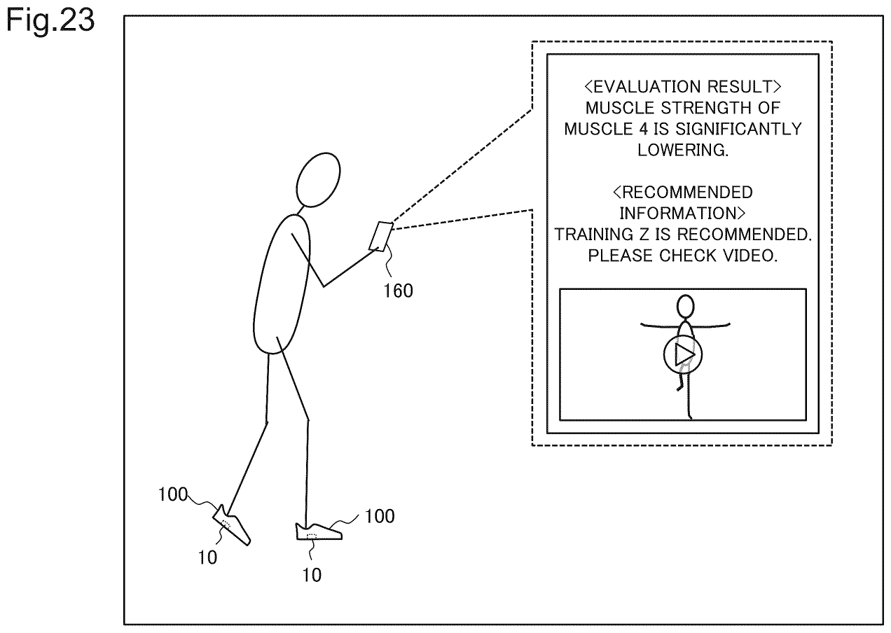

FIG. 23 is another example in which information corresponding to the evaluation result of the muscle strength is displayed on the screen of the mobile terminal 160. In the example of FIG. 23, an evaluation result “muscle strength of muscle 4 is significantly lowering” is displayed on the screen of the mobile terminal 160. Furthermore, in the example of FIG. 23, recommended information corresponding to the evaluation result “training Z is recommended. Please see the following video” is displayed on the display unit of the mobile terminal 160 according to the evaluation result of the muscle strength. On the screen of the mobile terminal 160, a video related to training leading to an increase in the muscle strength of the muscle 4 that is significantly lowering is displayed. The user who has confirmed the information displayed on the display unit of the mobile terminal 160 can practice training leading to an increase in muscle strength of the muscle 4 by exercising with reference to the video of the training Z according to the recommended information.

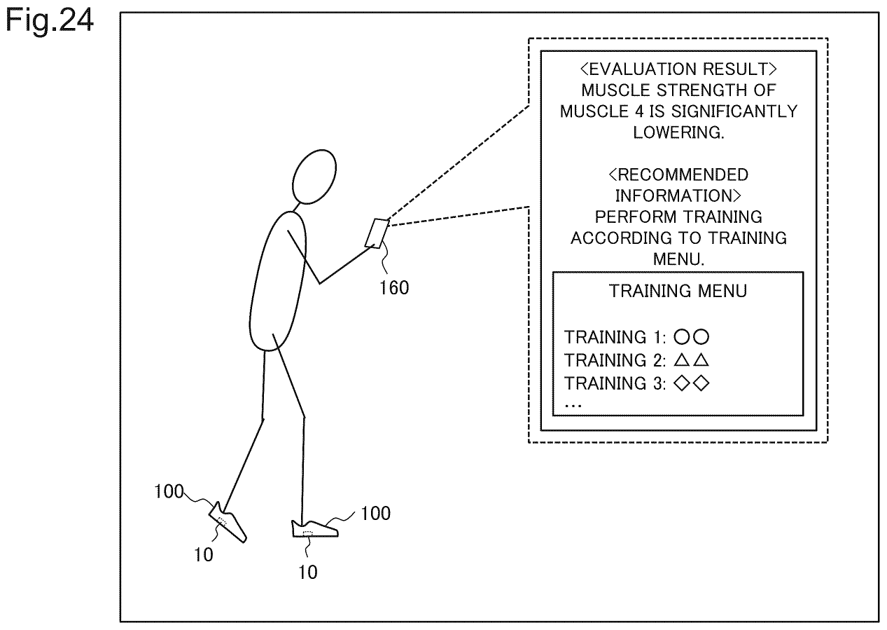

FIG. 24 illustrates still another example in which information corresponding to the evaluation result of muscle strength is displayed on the screen of the mobile terminal 160. In the example of FIG. 24, an evaluation result “muscle strength of the muscle 4 is significantly lowering” is displayed on the screen of the mobile terminal 160. In the example of FIG. 24, recommended information corresponding to the evaluation result “Please perform training according to the following training menu” is displayed on the display unit of the mobile terminal 160 according to the evaluation result of the muscle strength. On the screen of the mobile terminal 160, a training menu leading to an increase in the muscle strength of the muscle 4 that is significantly lowering is displayed. The user who has confirmed the information displayed on the display unit of the mobile terminal 160 can practice training leading to an increase in muscle strength of the muscle 4 by exercising with reference to the training menu according to the recommended information.

As described above, the muscle strength evaluation system of the present example embodiment includes the gait measuring device and the muscle strength evaluation device. The gait measuring device includes a sensor and a feature quantity data generation unit. The sensor includes an acceleration sensor and an angular velocity sensor. The sensor measures a spatial acceleration using an acceleration sensor. The sensor measures a spatial angular velocity using an angular velocity sensor. The sensor uses the measured spatial acceleration and spatial angular velocity to generate sensor data related to the movement of the foot. The sensor outputs the generated sensor data to the feature quantity data generation unit. The feature quantity data generation unit acquires time-series data of the sensor data related to the movement of the foot. The feature quantity data generation unit extracts walking waveform data for one gait cycle from the time-series data of the sensor data. The feature quantity data generation unit normalizes the extracted walking waveform data. The feature quantity data generation unit extracts a feature quantity used for estimation of the muscle strength of the evaluation target muscle from the normalized walking waveform data. The feature quantity data generation unit generates feature quantity data including the extracted feature quantity. The feature quantity data generation unit outputs the generated feature quantity data to the muscle strength evaluation device.

The muscle strength evaluation device includes a data acquisition unit, a storage unit, an evaluation unit, and an output unit. The data acquisition unit acquires feature quantity data including a feature quantity used for estimation of muscle strength of an evaluation target muscle related to a fall risk, the feature quantity data being extracted from the sensor data related to the movement of a user's foot.

The storage unit stores an estimation model that outputs a muscle strength index of an evaluation target muscle according to an input of the feature quantity data. The evaluation unit inputs the acquired feature quantity data to the estimation model, and evaluates the muscle strength of the evaluation target muscle of the user according to the muscle strength index output from the estimation model. The output unit outputs information regarding an evaluation result related to the muscle strength of the evaluation target muscle of the user.

The muscle strength evaluation system of the present example embodiment evaluates the muscle strength of the evaluation target muscle of the user by using the feature quantity extracted from the sensor data related to the movement of the user's foot. Therefore, according to the present example embodiment, the muscle strength of the muscle related to the risk of falling can be evaluated according to the gait in daily life without using an instrument for evaluating the muscle strength.

In one aspect of the present example embodiment, the storage unit stores a physical ability estimation model and a muscle strength estimation model. The physical ability estimation model outputs a physical ability score according to an input of a feature quantity used to estimate a physical ability score related to a fall risk. The muscle strength estimation model outputs the muscle strength score of the evaluation target muscle according to the input of the physical ability score. The data acquisition unit acquires a feature quantity extracted from the walking waveform data generated using the time-series data of the sensor data and used for estimation of the physical ability score. The evaluation unit inputs the acquired feature quantity to the physical ability estimation model. The evaluation unit inputs the physical ability score output from the estimation model to the muscle strength estimation model. The evaluation unit evaluates the muscle strength of the evaluation target muscle of the user according to the muscle strength score output from the muscle strength estimation model. According to the present aspect, the muscle strength of the evaluation target muscle of the user can be evaluated using the estimation model including the physical ability estimation model and the muscle strength estimation model.

In one aspect of the present example embodiment, the storage unit stores a physical ability estimation model and a muscle strength estimation model. The physical ability estimation model is generated by machine learning, with respect to a plurality of subjects, using teacher data in which a feature quantity used for estimation of the physical ability score is an explanatory variable and physical ability scores for a plurality of subjects are objective variables. The muscle strength estimation model is generated by machine learning, with respect to a plurality of subjects, using teacher data in which a physical ability scores are explanatory variables, and a muscle strength score of the evaluation target muscle for a plurality of subjects is an objective variable. The evaluation unit inputs, to the physical ability estimation model, the feature quantity acquired for the user and used for estimation of the physical ability score. The evaluation unit inputs the physical ability score output from the estimation model to the muscle strength estimation model. The evaluation unit evaluates the muscle strength of the evaluation target muscle of the user according to the muscle strength score output from the muscle strength estimation model. According to the present aspect, the muscle strength of the evaluation target muscle of the user can be evaluated using the estimation model in which the teacher data for the plurality of subjects is machine learned.

In one aspect of the present example embodiment, the storage unit stores an estimation model machine learned using explanatory variables including attribute data of a plurality of subjects. The evaluation unit inputs the feature quantity and the attribute data regarding the user to the estimation model, and estimates the muscle strength of the user according to the muscle strength index of the user output from the estimation model. In the present aspect, the muscle strength is estimated including attribute data that affects the muscle strength of the evaluation target muscle. Therefore, according to the present aspect, the muscle strength of the evaluation target muscle of the user can be evaluated with higher accuracy according to the attribute of the user.

In one aspect of the present example embodiment, the storage unit stores a physical ability estimation model and a muscle strength estimation model. The physical ability estimation model is generated by machine learning using teacher data in which a feature quantity used for estimation of a physical ability score of a physical ability related to a fall risk for a plurality of subjects is an explanatory variable and a physical ability score for the plurality of subjects is an objective variable. The physical ability related to the risk of falling is at least one of the five items of the total muscle strength of the whole body, the dynamic balance, the lower limb muscle strength, the mobility, and the static balance. The muscle strength estimation model is generated by machine learning using teacher data in which a physical ability score related to at least one physical ability of the physical abilities of the five items is as an explanatory variable for a plurality of subjects and a muscle strength score of an evaluation target muscle for the plurality of subjects is an objective variable. The data acquisition unit acquires a feature quantity extracted from the walking waveform data and used for estimation of the physical ability score. The evaluation unit inputs the feature quantity acquired regarding the user to the physical ability estimation model. The evaluation unit inputs the physical ability score output from the physical ability estimation model to the muscle strength estimation model. The evaluation unit evaluates the muscle strength of the evaluation target muscle of the user according to the muscle strength score output from the muscle strength estimation model. According to the present aspect, the muscle strength of the evaluation target muscle of the user can be evaluated according to the physical ability associated with the fall risk.

In one aspect of the present example embodiment, the storage unit stores the specific gravity of the evaluation target muscle related to the physical ability of five items and the specific gravity coefficient of the evaluation target muscle determined in advance by the physical ability test of the five items. The evaluation unit calculates a muscle strength score of the evaluation target muscle by using a product of a specific gravity of the evaluation target muscle in the physical ability related to the evaluation target muscle, a specific gravity coefficient of the evaluation target muscle in the physical ability related to the evaluation target muscle, and a physical ability score related to the physical ability related to the evaluation target muscle. The evaluation unit evaluates the muscle strength of the evaluation target muscle according to the calculated muscle strength score of the evaluation target muscle. According to the present aspect, the muscle strength of the evaluation target muscle can be estimated using the specific gravity and the specific gravity coefficient of the evaluation target muscle in the physical ability related to the evaluation target muscle.

In one aspect of the present example embodiment, the muscle strength evaluation device is mounted on a terminal device having a screen visually recognizable by the user. For example, the muscle strength evaluation device displays information regarding the muscle strength of the evaluation target muscle estimated according to the sensor data on the screen of the terminal device. For example, the muscle strength evaluation device displays recommended information corresponding to the muscle strength of the evaluation target muscle estimated according to the feature quantity extracted from the sensor data on the screen of the terminal device. For example, the muscle strength evaluation device displays a video related to training for training the evaluation target muscle on the screen of the terminal device as recommended information corresponding to the muscle strength estimated according to the feature quantity extracted from the sensor data. For example, the muscle strength evaluation device displays a training menu for training the evaluation target muscle on the screen of the terminal device as recommended information corresponding to the muscle strength estimated according to the feature quantity extracted from the sensor data. According to the present aspect, the user can confirm the information corresponding to the muscle strength of the user by displaying the information regarding the muscle strength of the evaluation target muscle estimated according to the feature quantity extracted from the sensor data on the screen visually recognizable by the user.

### Second Example Embodiment

Next, a machine learning system according to a second example embodiment will be described with reference to the drawings. The machine learning system of the present example embodiment generates an estimation model for estimating muscle strength according to an input of a feature quantity by machine learning using the feature quantity data extracted from the sensor data measured by a gait measuring device.

### (Configuration)

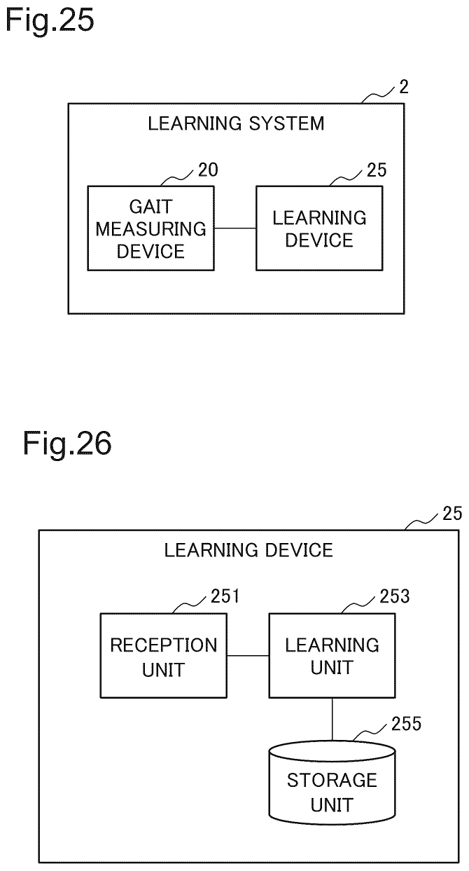

FIG. 25 is a block diagram illustrating an example of a configuration of the machine learning system 2 according to the present example embodiment. The machine learning system 2 includes a gait measuring device 20 and a machine learning device 25. The gait measuring device 20 and the machine learning device 25 may be connected by wire or wirelessly. The gait measuring device 20 and the machine learning device 25 may be configured by a single device. In addition, the machine learning system 2 may be configured only by the machine learning device 25, excluding the gait measuring device 20 from the configuration of the machine learning system 2. Although only one gait measuring device 20 is illustrated in FIG. 25, one (two in total) gait measuring device 20 may be arranged on each of the left and right feet. Furthermore, the machine learning device 25 may not be connected to the gait measuring device 20 and may be configured to execute machine learning using the feature quantity data generated in advance by the gait measuring device 20 and stored in the database.

The gait measuring device 20 is installed on at least one of the left and right legs. The gait measuring device 20 has the same configuration as the gait measuring device 10 of the first example embodiment. The gait measuring device 20 includes an acceleration sensor and an angular velocity sensor. The gait measuring device 20 converts the measured physical quantity into digital data (also referred to as sensor data). The gait measuring device 20 generates normalized walking waveform data for one gait cycle from the time-series data of the sensor data. The gait measuring device 20 generates feature quantity data used for estimation of the muscle strength of the evaluation target muscle related to the fall risk. For example, the gait measuring device 20 generates feature quantity data used for estimation of scores regarding five items of the total muscle strength of the whole body, the dynamic balance, the lower limb muscle strength, the mobility, and the static balance. The gait measuring device 20 transmits the generated feature quantity data to the machine learning device 25. The gait measuring device 20 may be configured to transmit the feature quantity data to a database (not illustrated) accessed by the machine learning device 25. The feature quantity data accumulated in the database is used for machine learning by the machine learning device 25.

The machine learning device 25 receives the feature quantity data extracted from the walking waveform data of the plurality of subjects from the gait measuring device 20. When using the feature quantity data accumulated in the database (not illustrated), the machine learning device 25 receives the feature quantity data from the database. The machine learning device 25 executes machine learning using the received feature quantity data. For example, the machine learning device 25 learns teacher data in which the feature quantity data for estimating scores of five items related to the fall risk is an explanatory variable and scores of five items corresponding to the feature quantity data are objective variables. For example, the machine learning device 25 machine learns teacher data in which at least one of the scores of five items related to the fall risk is an explanatory variable and the muscle strength score of the evaluation target muscle is an objective variable. The algorithm of machine learning executed by the machine learning device 25 is not particularly limited. The machine learning device 25 generates an estimation model machine learned using teacher data related to a plurality of subjects. The machine learning device 25 stores the generated estimation model. The estimation model machine learned by the machine learning device 25 may be stored in a storage device outside the machine learning device 25.

### [Machine Learning Device]

Next, details of the machine learning device 25 will be described with reference to the drawings. FIG. 26 is a block diagram illustrating an example of a detailed configuration of the machine learning device 25. The machine learning device 25 includes a reception unit 251, a machine learning unit 253, and a storage unit 255.

The reception unit 251 receives the feature quantity data from the gait measuring device 20. The reception unit 251 outputs the received feature quantity data to the machine learning unit 253. The reception unit 251 may receive the feature quantity data from the gait measuring device 20 via a wire such as a cable, or may receive the feature quantity data from the gait measuring device 20 via wireless communication. For example, the reception unit 251 is configured to receive the feature quantity data from the gait measuring device 20 via a wireless communication function (not illustrated) conforming to a standard such as Bluetooth (registered trademark) or WiFi (registered trademark). The communication function of the reception unit 251 may conform to a standard other than Bluetooth (registered trademark) or WiFi (registered trademark).

The machine learning unit 253 acquires the feature quantity data from the reception unit 251. The machine learning unit 253 executes machine learning using the acquired feature quantity data. The machine learning unit 253 generates a physical ability estimation model that outputs scores of five items related to the fall risk according to the input of the feature quantity data by machine learning using the feature quantity data. For example, the machine learning unit 253 machine learns, as teacher data, a data set in which the feature quantity data extracted from the sensor data measured according to the movement of the foot of the subject is an explanatory variable and scores of five items related to the fall risk are objective variables. In addition, the machine learning unit 253 generates the muscle strength estimation model that outputs the muscle strength score of the evaluation target muscle according to the input of the scores of five items by machine learning using the scores of five items related to the fall risk. For example, the machine learning unit 253 machine learns teacher data in which at least one of the scores of five items is an explanatory variable and the muscle strength score of the evaluation target muscle is an objective variable. For example, the machine learning unit 253 generates an estimation model corresponding to the attribute data. The machine learning unit 253 stores estimation models machine learned for a plurality of subjects in the storage unit 255.

For example, the machine learning unit 253 executes machine learning using a linear regression algorithm. For example, the machine learning unit 253 executes machine learning using a support vector machine (SVM) algorithm. For example, the machine learning unit 253 executes machine learning using a Gaussian process regression (GPR) algorithm. 1. For example, the machine learning unit 253 executes machine learning using a random forest (RF) algorithm. For example, the machine learning unit 253 may execute unsupervised machine learning of classifying a subject who is a generation source of the feature quantity data according to the feature quantity data. The algorithm of machine learning executed by the machine learning unit 253 is not particularly limited.

The machine learning unit 253 may execute machine learning using the walking waveform data for one gait cycle as an explanatory variable. For example, the machine learning unit 253 executes supervised machine learning in which the acceleration in the three axes direction, the angular velocity around the three axes, and the walking waveform data of the angle (posture angle) around the three axes are explanatory variables and the correct value of the muscle strength index is used as an objective variable. For example, when the walking phase is set by 1% interval in the gait cycle of 0 to 100%, the machine learning unit 253 machine learns using 909 explanatory variables.

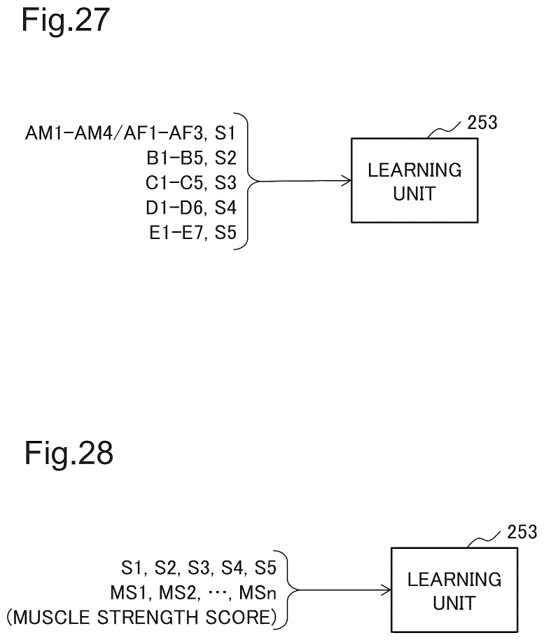

FIG. 27 is a conceptual diagram for describing machine learning for generating a physical ability estimation model for estimating a score of a physical ability related to a fall risk. FIG. 27 is a conceptual diagram illustrating an example of causing the machine learning unit 253 to machine learn a data set of feature quantities as explanatory variables and scores as objective variables corresponding to the feature quantities as teacher data. In machine learning of the model for estimating the total muscle strength score S1 of the male, a data set of the feature quantities AM1 to AM4 and the total muscle strength score S1 of the male is used as teacher data. In machine learning of the model for estimating the total muscle strength score S1 of the female, a data set of the feature quantities AF1 to AF3 and the total muscle strength score S1 of the female is used as teacher data. In the machine learning of the model for estimating the dynamic balance score S2, a data set of the feature quantities B1 to B5 and the dynamic balance score S2 is used as teacher data. In the machine learning of the model for estimating the lower limb muscle strength score S3, a data set of the feature quantities C1 to C4 and the lower limb muscle strength score S3 is used as teacher data. In the machine learning of the model for estimating the mobility score S4, a data set of the feature quantities D1 to D6 and the mobility score S4 is used as teacher data. In the machine learning of the model for estimating the static balance score S5, a data set of the feature quantities E1 to E7 and the static balance score S5 is used as teacher data. For example, the machine learning unit 253 machine learns data for a plurality of subjects, and generates a physical ability estimation model that outputs physical ability scores of five items related to the fall risk according to the input of the feature quantity extracted from the sensor data. In the machine learning for generating the physical ability estimation model, a plurality of estimation models included in the physical ability estimation model may be individually generated, or a plurality of estimation models may be collectively generated.

FIG. 28 is a conceptual diagram for describing machine learning for generating a muscle strength estimation model. FIG. 28 is a conceptual diagram illustrating an example of causing the machine learning unit 253 to machine learn a data set of scores of five items as explanatory variables and muscle strength scores as objective variables as teacher data (n is a natural number). In the example of FIG. 28, teacher data is used in which the total muscle strength score S1 of the whole body, the dynamic balance score S2, the lower limb muscle strength score S3, the mobility score S4, and the static balance score S5 are explanatory variables, and the muscle strength scores MS1 to MSn objective variables. For example, the machine learning unit 253 machine learns data for a plurality of subjects, and generates a muscle strength estimation model that outputs a muscle strength score (muscle strength index) according to an input of a feature quantity extracted from the sensor data.

The storage unit 255 stores an estimation model machine learned for a plurality of subjects and used for estimating the muscle strength of the evaluation target muscle. The estimation model stored in the storage unit 255 is used for estimation of the muscle strength by the muscle strength evaluation device 13 of the first example embodiment.

As described above, the machine learning system of the present example embodiment includes the gait measuring device and the machine learning device. The gait measuring device acquires time-series data of the sensor data regarding a movement of a foot. The gait measuring device extracts walking waveform data for one gait cycle from the time-series data of the sensor data, and normalizes the extracted walking waveform data. The gait measuring device extracts a feature quantity used for evaluation of an evaluation target muscle of the user from a walking phase cluster configured by at least one temporally continuous walking phase from the normalized walking waveform data. The gait measuring device generates feature quantity data including the extracted feature quantity. The gait measuring device outputs the generated feature quantity data to the machine learning device.

The machine learning device includes a reception unit, a machine learning unit, and a storage unit. The reception unit acquires the feature quantity data generated by the gait measuring device. The machine learning unit executes machine learning using the feature quantity data. The machine learning unit generates the estimation model that outputs the muscle strength of the evaluation target muscle according to the input of the feature quantity of the walking phase cluster extracted from the time-series data of the sensor data measured along with the walking of the user. The estimation model generated by the machine learning unit is stored in the storage unit.

The machine learning system of the present example embodiment generates an estimation model by using the feature quantity data measured by the gait measuring device. Therefore, according to the present aspect, it is possible to generate an estimation model that enables the muscle strength of the muscle related to the fall risk to be evaluated according to the gait in daily life without using an instrument for evaluating the muscle strength.

In one aspect of the present example embodiment, the gait measuring device extracts, from the normalized walking waveform data, a feature quantity related to at least one of five items of the total muscle strength of the whole body, the dynamic balance, the lower limb muscle strength, the mobility, and the static balance. For example, the machine learning unit generates an estimation model (physical ability estimation model) by machine learning using teacher data having a feature quantity related to at least one of the five items as an explanatory variable and a score of the five items corresponding to the feature quantity used as the explanatory variable as an objective variable. The machine learning unit generates the muscle strength estimation model that outputs the muscle strength score of the evaluation target muscle according to the input of the score related to at least one of the five items. According to the present aspect, the physical ability estimation model can be generated that enables the score related to the five items to be estimated according to the input of the feature quantity related to the five items. Furthermore, according to the present aspect, a muscle strength estimation model can be generated that enables the muscle strength of the evaluation target muscle to be evaluated according to the input of the score related to the five items.

### Third Example Embodiment

Next, a muscle strength evaluation device according to a third example embodiment will be described with reference to the drawings. The muscle strength evaluation device of the present example embodiment has a simplified configuration of the muscle strength evaluation device included in the muscle strength evaluation system of the first example embodiment.

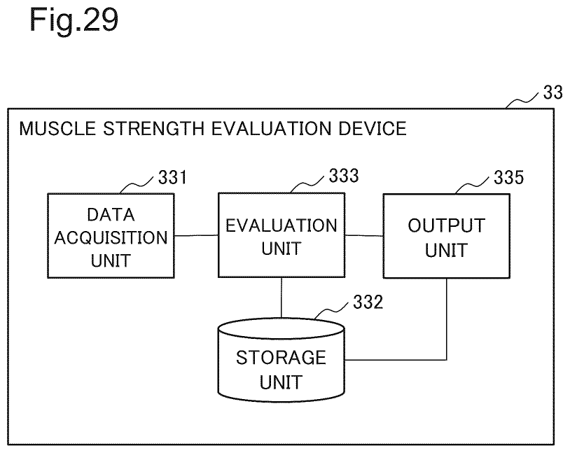

FIG. 29 is a block diagram showing an example of a configuration of a muscle strength evaluation device 33 according to the present example embodiment. The muscle strength evaluation device 33 includes a data acquisition unit 331, a storage unit 332, an evaluation unit 333, and an output unit 335.

The data acquisition unit 331 acquires feature quantity data including a feature quantity used for estimation of the muscle strength of an evaluation target muscle related to a fall risk, the feature quantity data being extracted from the sensor data related to the movement of a user's foot. The storage unit 332 stores an estimation model that outputs a muscle strength index of an evaluation target muscle according to an input of the feature quantity data. The evaluation unit 333 inputs the acquired feature quantity data to the estimation model, and evaluates the muscle strength of the evaluation target muscle of the user according to the muscle strength index output from the estimation model. The output unit 335 outputs information regarding an evaluation result related to the muscle strength of the evaluation target muscle of the user.

As described above, in the present example embodiment, the muscle strength of the evaluation target muscle of the user is evaluated using the feature quantity extracted from the sensor data related to the movement of the user's foot. Therefore, according to the present example embodiment, the muscle strength of the muscle related to the risk of falling can be evaluated according to the gait in daily life without using an instrument for evaluating the muscle strength.

### (Hardware)

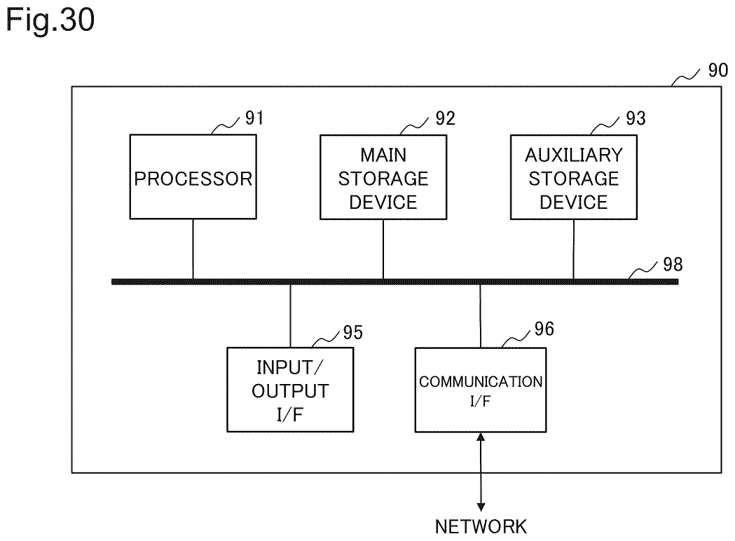

Here, a hardware configuration for executing control and process according to each example embodiment of the present disclosure will be described using an information processing device 90 of FIG. 30 as an example. The information processing device 90 in FIG. 30 is a configuration example for executing control and process of each example embodiment, and does not limit the scope of the present disclosure.

As illustrated in FIG. 30, the information processing device 90 includes a processor 91, a main storage device 92, an auxiliary storage device 93, an input/output interface 95, and a communication interface 96. In FIG. 30, the interface is abbreviated as an interface (I/F). The processor 91, the main storage device 92, the auxiliary storage device 93, the input/output interface 95, and the communication interface 96 are connected to each other via a bus 98 in such a way as to be able to communicate data. In addition, the processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, a configuration of using a software program installed in the information processing device 90 may be adopted. The processor 91 executes control and process according to each example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). In addition, a nonvolatile memory such as a magnetoresistive random access memory (MRAM) may be configured/added as the main storage device 92.

The auxiliary storage device 93 stores various data such as programs. The auxiliary storage device 93 is achieved by a local disk such as a hard disk or a flash memory. Various data may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to an external system or device through a network such as the Internet or an intranet based on a standard or a specification. The input/output interface 95 and the communication interface 96 may be shared as an interface to connect to an external device.

Input devices such as a keyboard, a mouse, and a touch panel may be connected to the information processing device 90 as necessary. These input devices are used to input information and settings. When the touch panel is used as the input device, the display screen of the display device may also serve as the interface of the input device. Data communication between the processor 91 and the input device may be mediated by the input/output interface 95.

Furthermore, the information processing device 90 may be provided with a display device for displaying information. In a case where a display device is provided, the information processing device 90 preferably includes a display control device (not illustrated) for controlling display of the display device. The display device may be connected to the information processing device 90 via the input/output interface 95.

Furthermore, the information processing device 90 may be provided with a drive device. The drive device mediates reading of data and a program from a recording medium, writing of a processing result of the information processing device 90 to the recording medium, and the like between the processor 91 and the recording medium (program recording medium). The display device may be connected to the information processing device 90 via the input/output interface 95.

The above is an example of a hardware configuration for enabling control and process according to each example embodiment of the present invention. The hardware configuration of FIG. 30 is an example of a hardware configuration for executing control and process according to each example embodiment, and does not limit the scope of the present invention. In addition, a program for causing a computer to execute control and process according to each example embodiment is also included in the scope of the present invention. Furthermore, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. Furthermore, the recording medium may be achieved by a magnetic recording medium such as a flexible disk, or another recording medium. When a program executed by the processor is recorded in a recording medium, the recording medium corresponds to a program recording medium.

The components of each example embodiment may be arbitrarily combined. In addition, the components of each example embodiment may be achieved by software or may be achieved by a circuit.

Although the present invention has been described with reference to the example embodiments, the present invention is not limited to the above example embodiments. Various modifications that can be understood by those skilled in the art can be made to the configuration and details of the present invention within the scope of the present invention.

Some or all of the above example embodiments may be described as the following supplementary notes, but are not limited to the following.

### (Supplementary Note 1)

A muscle strength evaluation device including:
a data acquisition unit that acquires feature quantity data including a feature quantity used for estimation of a muscle strength of an evaluation target muscle related to a fall risk, the feature quantity data being extracted from sensor data related to movement of a user's foot:
a storage unit that stores an estimation model that outputs a muscle strength index of the evaluation target muscle according to an input of the feature quantity data:
an evaluation unit that inputs the acquired feature quantity data to the estimation model and evaluate a muscle strength of the evaluation target muscle of the user according to the muscle strength index output from the estimation model; and
an output unit that outputs information regarding an evaluation result related to the muscle strength of the evaluation target muscle of the user.

### (Supplementary Note 2)

The muscle strength evaluation device according to supplementary note 1, wherein
the storage unit stores
a physical ability estimation model that outputs a physical ability score according to an input of a feature quantity used for estimation of the physical ability score related to a fall risk, and
a muscle strength estimation model that outputs a muscle strength score of the evaluation target muscle according to an input of the physical ability score,
the data acquisition unit
acquires a feature quantity used for estimation of the physical ability score extracted from walking waveform data generated using time-series data of the sensor data, and
the evaluation unit
inputs the acquired feature quantity to the physical ability estimation model,
inputs the physical ability score output from the estimation model to the muscle strength estimation model, and
evaluates a muscle strength of the evaluation target muscle of the user according to the muscle strength score output from the muscle strength estimation model.

### (Supplementary Note 3)

The muscle strength evaluation device according to supplementary note 2, wherein
the storage unit stores
the physical ability estimation model generated by machine learning, with respect to a plurality of subjects, using teacher data in which a feature quantity used for estimation of the physical ability score is an explanatory variable and the physical ability score for the plurality of subjects is an objective variable, and
the muscle strength estimation model generated by machine learning, with respect to a plurality of the subjects, using teacher data in which the physical ability score is an explanatory variable and the muscle strength score of the evaluation target muscle for the plurality of subjects is an objective variable, and
the evaluation unit
inputs a feature quantity acquired with respect to the user and used for estimation of the physical ability score to the physical ability estimation model,
inputs the physical ability score output from the estimation model to the muscle strength estimation model, and
evaluates a muscle strength of the evaluation target muscle of the user according to the muscle strength score output from the muscle strength estimation model.

### (Supplementary Note 4)

The muscle strength evaluation device according to supplementary note 3, wherein
the storage unit stores the estimation model machine learned using an explanatory variable including attribute data of a plurality of the subjects, and
the evaluation unit inputs a feature quantity related to the user and the attribute data to the estimation model, and evaluates the muscle strength of the evaluation target muscle of the user according to the muscle strength index of the user output from the estimation model.

### (Supplementary Note 5)

The muscle strength evaluation device according to supplementary note 3 or 4, wherein
the storage unit stores
the physical ability estimation model generated by machine learning, with respect to a plurality of the subjects, using teacher data in which a feature quantity used for estimation of the physical ability score regarding at least one physical ability of the physical abilities of five items of total muscle strength of the whole body, dynamic balance, lower limb muscle strength, mobility, and static balance is an explanatory variable, and the physical ability score regarding the physical ability of at least one of the five items for the plurality of the subjects is an objective variable, and
the muscle strength estimation model generated by machine learning, with respect to a plurality of the subjects, using teacher data in which the physical ability score related to at least one physical ability of the physical abilities of the five items is an explanatory variable, and the muscle strength score of the evaluation target muscle for a plurality of the subjects is an objective variable,
the data acquisition unit
acquires a feature quantity extracted from the walking waveform data and used for estimation of the physical ability score, and
the evaluation unit
inputs a feature quantity acquired with respect to the user to the physical ability estimation model,
inputs the physical ability score output from the physical ability estimation model to the muscle strength estimation model, and
evaluates the muscle strength of the evaluation target muscle of the user according to the muscle strength score output from the muscle strength estimation model.

### (Supplementary Note 6)

The muscle strength evaluation device according to supplementary note 5, wherein
the storage unit
stores a specific gravity of the evaluation target muscle related to the physical abilities of the five items and a specific gravity coefficient of the evaluation target muscle determined in advance by tests of the physical abilities of the five items, and
the evaluation unit
calculates the muscle strength score of the evaluation target muscle by using a product of a specific gravity of the evaluation target muscle in the physical ability related to the evaluation target muscle, a specific gravity coefficient of the evaluation target muscle in the physical ability related to the evaluation target muscle, and the physical ability score related to the physical ability for the evaluation target muscle, and
evaluates the muscle strength of the evaluation target muscle according to the calculated muscle strength score of the evaluation target muscle.

### (Supplementary Note 7)

A muscle strength evaluation system including:
the muscle strength evaluation device according to any one of supplementary notes 1 to 6; and
a gait measuring device including a sensor that is installed on footwear of a user who is an evaluation target of muscle strength, measures a spatial acceleration and a spatial angular velocity, generates sensor data regarding a movement of a foot using the measured spatial acceleration and spatial angular velocity, and outputs the generated sensor data; and a feature quantity data generation unit that extracts walking waveform data for one gait cycle from time-series data of the sensor data, normalizes the extracted walking waveform data, extracts a feature quantity used for estimation of the muscle strength of an evaluation target muscle from the normalized walking waveform data, generates feature quantity data including the extracted feature quantity, and outputs the generated feature quantity data to the muscle strength evaluation device.

### (Supplementary Note 8)

The muscle strength evaluation system according to supplementary note 7, wherein
the muscle strength evaluation device
is implemented in a terminal device having a screen visually recognizable by the user, and
displays information regarding the muscle strength of the evaluation target muscle evaluated according to the feature quantity extracted from the sensor data on a screen of the terminal device.

### (Supplementary Note 9)

The muscle strength evaluation system according to supplementary note 8, wherein the muscle strength evaluation device
displays recommended information corresponding to the muscle strength of the evaluation target muscle evaluated according to the feature quantity extracted from the sensor data on a screen of the terminal device.

### (Supplementary Note 10)

The muscle strength evaluation system according to supplementary note 9, wherein
the muscle strength evaluation device displays a video related to training for training the evaluation target muscle on a screen of the terminal device, as the recommended information corresponding to the muscle strength of the evaluation target muscle evaluated according to the feature quantity extracted from the sensor data.

### (Supplementary Note 11)

The muscle strength evaluation system according to supplementary note 9 or 10, wherein the muscle strength evaluation device
displays a training menu for training a body part related to the muscle strength of the evaluation target muscle on a screen of the terminal device, as the recommended information corresponding to the muscle strength of the evaluation target muscle evaluated according to the feature quantity extracted from the sensor data.

### (Supplementary Note 12)

A muscle strength evaluation method in which a computer is configured to:
acquire feature quantity data including a feature quantity used for estimation of muscle strength of an evaluation target muscle related to a fall risk, the feature quantity data being extracted from sensor data regarding a movement of a user's foot:
input the acquired feature quantity data to an estimation model that outputs a muscle strength index of the evaluation target muscle according to an input of the feature quantity data:
evaluate the muscle strength of the evaluation target muscle of the user according to the muscle strength index output from the estimation model; and
output information regarding an evaluation result related to a muscle strength of the evaluation target muscle of the user.

### (Supplementary Note 13)

A program for causing a computer to execute processes of:
acquiring feature quantity data including a feature quantity used for estimation of muscle strength of an evaluation target muscle related to a fall risk, the feature quantity data being extracted from sensor data regarding a movement of a user's foot;
inputting the acquired feature quantity data to an estimation model that outputs a muscle strength index of the evaluation target muscle according to an input of the feature quantity data;
evaluating the muscle strength of the evaluation target muscle of the user according to the muscle strength index output from the estimation model; and
outputting information regarding an evaluation result related to a muscle strength of the evaluation target muscle of the user.

### REFERENCE SIGNS LIST

1 muscle strength evaluation system
2 machine learning system
10, 20 gait measuring device
11 sensor
12 feature quantity data generation unit
13 muscle strength evaluation device
25 machine learning device
111 acceleration sensor
112 angular velocity sensor
121 acquisition unit
122 normalization unit
123 extraction unit
125 generation unit
127 feature quantity data output unit
130, 333 evaluation unit
131, 331 data acquisition unit
132, 332 storage unit
133 physical ability estimation unit
134 muscle strength evaluation unit
135, 335 output unit
251 reception unit
253 machine learning unit
255 storage unit

What is claimed is:

1. A muscle strength evaluation device comprising:
a storage configured to store an estimation model that outputs a muscle strength index of the evaluation target muscle according to an input of feature amount data used for estimating a muscle strength of an evaluation target muscle related to a fall risk;
a memory storing instructions; and
a processor connected to the memory and configured to execute the instructions to:
acquire feature quantity data including a feature quantity used for estimation of a muscle strength of an evaluation target muscle related to a fall risk, the feature quantity data being extracted from sensor data related to movement of a user's foot;
input the acquired feature quantity data to the estimation model and evaluate a muscle strength of the evaluation target muscle of the user according to the muscle strength index output from the estimation model; and
output information regarding an evaluation result related to the muscle strength of the evaluation target muscle of the user.

2. The muscle strength evaluation device according to claim 1, wherein the storage stores
a physical ability estimation model that outputs a physical ability score according to an input of a feature quantity used for estimation of the physical ability score related to a fall risk, and
a muscle strength estimation model that outputs a muscle strength score of the evaluation target muscle according to an input of the physical ability score; and
the processor is configured to execute the instructions to acquire a feature quantity used for estimation of the physical ability score extracted from walking waveform data generated using time-series data of the sensor data,
input the acquired feature quantity to the physical ability estimation model,
input the physical ability score output from the estimation model to the muscle strength estimation model, and
evaluate a muscle strength of the evaluation target muscle of the user according to the muscle strength score output from the muscle strength estimation model.

3. The muscle strength evaluation device according to claim 2, wherein
the storage stores
the physical ability estimation model generated by machine learning, with respect to a plurality of subjects, using teacher data in which a feature quantity used for estimation of the physical ability score is an explanatory variable and the physical ability score for the plurality of subjects is an objective variable, and
the muscle strength estimation model generated by machine learning, with respect to a plurality of the subjects, using teacher data in which the physical ability score is an explanatory variable and the muscle strength score of the evaluation target muscle for the plurality of the subjects is an objective variable, and
the processor is configured to execute the instructions to
input a feature quantity acquired with respect to the user and used for estimation of the physical ability score to the physical ability estimation model,
input the physical ability score output from the estimation model to the muscle strength estimation model, and
evaluate a muscle strength of the evaluation target muscle of the user according to the muscle strength score output from the muscle strength estimation model.

4. The muscle strength evaluation device according to claim 3, wherein
the storage stores the estimation model machine learned using an explanatory variable including attribute data of a plurality of the subjects, and
the processor is configured to execute the instructions to
input a feature quantity related to the user and the attribute data to the estimation model, and
evaluate the muscle strength of the evaluation target muscle of the user according to the muscle strength index of the user output from the estimation model.

5. The muscle strength evaluation device according to claim 3, wherein
the storage stores
the physical ability estimation model generated by machine learning, with respect to a plurality of the subjects, using teacher data in which a feature quantity used for estimation of the physical ability score regarding at least one physical ability of the physical abilities of five items of total muscle strength of the whole body, dynamic balance, lower limb muscle strength, mobility, and static balance is an explanatory variable, and the physical ability score regarding the physical ability of at least one of the five items for the plurality of the subjects is an objective variable, and
the muscle strength estimation model generated by machine learning, with respect to a plurality of the subjects, using teacher data in which the physical ability score related to at least one physical ability of the physical abilities of the five items is an explanatory variable, and the muscle strength score of the evaluation target muscle for the plurality of the subjects is an objective variable, and
the processor is configured to execute the instructions to
acquire a feature quantity extracted from the walking waveform data and used for estimation of the physical ability score,
input a feature quantity acquired with respect to the user to the physical ability estimation model,
input the physical ability score output from the physical ability estimation model to the muscle strength estimation model, and
evaluate the muscle strength of the evaluation target muscle of the user according to the muscle strength score output from the muscle strength estimation model.

6. The muscle strength evaluation device according to claim 5, wherein
the storage stores a specific gravity of the evaluation target muscle related to the physical abilities of the five items and a specific gravity coefficient of the evaluation target muscle determined in advance by tests of the physical abilities of the five items, and
the processor is configured to execute the instructions to
calculate the muscle strength score of the evaluation target muscle by using a product of a specific gravity of the evaluation target muscle in the physical ability related to the evaluation target muscle, a specific gravity coefficient of the evaluation target muscle in the physical ability related to the evaluation target muscle, and the physical ability score related to the physical ability for the evaluation target muscle, and
evaluate the muscle strength of the evaluation target muscle according to the calculated muscle strength score of the evaluation target muscle.

7. A muscle strength evaluation system comprising:
the muscle strength evaluation device according to claim 1; and
a gait measuring device including
a sensor that is installed on footwear of a user who is an evaluation target of muscle strength, measures a spatial acceleration and a spatial angular velocity, generates sensor data regarding a movement of a foot using the measured spatial acceleration and spatial angular velocity, and outputs the generated sensor data,
a memory storing instructions, and
a processor connected to the memory and configured to execute the instructions to
extract walking waveform data for one gait cycle from time-series data of the sensor data,
normalize the extracted walking waveform data,
extract a feature quantity used for estimation of the muscle strength of an evaluation target muscle from the normalized walking waveform data,
generate feature quantity data including the extracted feature quantity, and
output the generated feature quantity data to the muscle strength evaluation device.

8. The muscle strength evaluation system according to claim 7, wherein
the muscle strength evaluation device is implemented in a terminal device having a screen visually recognizable by the user, and
the muscle strength evaluation device is configured to
display information regarding the muscle strength of the evaluation target muscle evaluated according to the feature quantity extracted from the sensor data on a screen of the terminal device.

9. The muscle strength evaluation system according to claim 8, wherein
the processer of the muscle strength evaluation device is configured to execute the instructions to
display recommended information corresponding to the muscle strength of the evaluation target muscle evaluated according to the feature quantity extracted from the sensor data on a screen of the terminal device.

10. The muscle strength evaluation system according to claim 9, wherein
the processer of the muscle strength evaluation device is configured to execute the instructions to
display a video related to training for training the evaluation target muscle on a screen of the terminal device, as the recommended information corresponding to the muscle strength of the evaluation target muscle evaluated according to the feature quantity extracted from the sensor data.

11. The muscle strength evaluation system according to claim 9, wherein
the processer of the muscle strength evaluation device is configured to execute the instructions to
display a training menu for training the evaluation target muscle on a screen of the terminal device, as the recommended information corresponding to the muscle strength of the evaluation target muscle evaluated according to the feature quantity extracted from the sensor data.

12. A muscle strength evaluation method in which a computer is configured to:
acquire feature quantity data including a feature quantity used for estimation of muscle strength of an evaluation target muscle related to a fall risk, the feature quantity data being extracted from sensor data regarding a movement of a user's foot;
input the acquired feature quantity data to an estimation model that outputs a muscle strength index of the evaluation target muscle according to an input of the feature quantity data;
evaluate the muscle strength of the evaluation target muscle of the user according to the muscle strength index output from the estimation model; and
output information regarding an evaluation result related to a muscle strength of the evaluation target muscle of the user.

13. A non-transient recording medium recorded with a program for causing a computer to execute:
processes of acquiring feature quantity data including a feature quantity used for estimation of muscle strength of an evaluation target muscle related to a fall risk, the feature quantity data being extracted from sensor data regarding a movement of a user's foot;
processes of inputting the acquired feature quantity data to an estimation model that outputs a muscle strength index of the evaluation target muscle according to an input of the feature quantity data;
processes of evaluating the muscle strength of the evaluation target muscle of the user according to the muscle strength index output from the estimation model; and
processes of outputting information regarding an evaluation result related to a muscle strength of the evaluation target muscle of the user.

14. The static balance estimation system according to claim 9, wherein
the processor of the mobility estimation device is configured to execute the instructions to
cause the recommendation information that supports the user for making decision about taking an action to be displayed on the screen of the terminal device.
